SEQUENCE LISTING (21 sequences)

SEQ ID NO: 1 - protein, 34 aa, synthetic construct
AWVFFIPFIMLTTFTVLNLFIGIIVDAMAITKEQ

SEQ ID NO: 2 - protein, 34 aa, synthetic construct
YMIFFVLVIFLGSFYLINLILAVVAMAYEEQNQA

SEQ ID NO: 3 - protein, 34 aa, synthetic construct
CLTVFMMVMVIGNLVVLNLFLALLLSSFSADNLA

SEQ ID NO: 4 - protein, 34 aa, synthetic construct
MYLYFVIFIIFGSFFTLNLFIGVIIDNFNQQKKK

SEQ ID NO: 5 - protein, 34 aa, synthetic construct
GIFFFVSYIIISFLVVVNMYIAVILENFSVATEE

SEQ ID NO: 6 - protein, 34 aa, synthetic construct; identical to SEQ ID NO: 2

SEQ ID NO: 7 - protein, 34 aa, synthetic construct
CLIVFMMVMVIGNLVVLNLFLALLLSSFSADNLA

SEQ ID NO: 8 - protein, 34 aa, synthetic construct
MYIYFVIFIIFGSFFTLNLFIGVIIDNFNQQKKK

SEQ ID NO: 9 - protein, 34 aa, synthetic construct
GIFFFVSYIIISFLIVVNMYIAIILENFSVATEE

SEQ ID NO: 10 - protein, 34 aa, synthetic construct; identical to SEQ ID NO: 2

SEQ ID NO: 11 - protein, 34 aa, synthetic construct
CLIVYMMVMVIGNLVVLNLFLALLLSSFSSDNLT

SEQ ID NO: 12 - protein, 34 aa, synthetic construct
MYIYFVVFIIFGSFFTLNLFIGVIIDNFNQQKKK

SEQ ID NO: 13 - protein, 34 aa, synthetic construct
GIFYFVSYIIISFLVVVNMYIAVILENFSVATEE

SEQ ID NO: 14 - protein, 34 aa, synthetic construct
YMIFFVVVIFLGSFYLVNLILAVVTMAYEEQNQA

SEQ ID NO: 15 - protein, 34 aa, synthetic construct
CLILFLTVMVLGNLVVLNLFIALLLNSFSADNLT

SEQ ID NO: 16 - protein, 34 aa, synthetic construct
MYLYFVIFIIFGGFFTLNLFVGVIIDNFNQQKKK

SEQ ID NO: 17 - protein, 34 aa, synthetic construct
GIIFFTTYIIISFLIMVNMYIAVILENFNVATEE

SEQ ID NO: 18 - protein, 34 aa, synthetic construct
SVFFFIVVIFLGSFYLINLTLAVVTMAYEEQNKN

SEQ ID NO: 19 - protein, 34 aa, synthetic construct
CVIVFILITVIGKLVVLNLFIALLLNSFSNEERN

SEQ ID NO: 20 - protein, 34 aa, synthetic construct
GYIYFVVFIIFGSFFTLNLFIGVIIDNFNQQQKK

SEQ ID NO: 21 - protein, 34 aa, synthetic construct
ATSYFVSYIIISFLIVVNMYIAVILENFNTATEE

(19) United States
(12) Patent Application Publication
(10) Pub. No.: US 2023/0141863 A1
(43) Pub. Date: May 11, 2023

(54) Small Molecule Inhibitors Targeting the Sodium Channel Tamoxifen Receptor and Uses Thereof

(71) Applicant: Northwestern University, Evanston, IL (US)

(72) Inventors: Paul Gregory DeCaen, Chicago, IL (US); Thuy Vien, Chicago, IL (US)

(21) Appl. No.: 18/054,527

(22) Filed: Nov. 10, 2022

Related U.S. Application Data
(60) Provisional application No. 63/263,882, filed on Nov. 10, 2021.

(51) Int. Cl. A61K 31/138; A61P 25/04; A61K 9/00

(52) U.S. Cl. CPC A61K 31/138; A61P 25/04; A61K 9/0053

(57) ABSTRACT

Disclosed are methods of treating diseases or disorders associated with the voltage-gated sodium channels (Naᵥs). The disclosed methods may be utilized to treat diseases or disorders associated with pain in a subject, including nociceptive pain, chronic pain, neuropathic pain, and inflammatory pain. Also disclosed are inhibitors of Naᵥs that specifically inhibit voltage-gated sodium channel function in a state-dependent manner. The disclosed compounds may also be used in pharmaceutical compositions and methods for treatment of pain associated with Naᵥ activity.

### CROSS-REFERENCE TO RELATED APPLICATIONS

This application claims benefit of priority to U.S. Patent Application Ser. No. 63/263,882, filed Nov. 10, 2021, the contents of which are incorporated herein by reference.

### REFERENCE TO AN ELECTRONIC SEQUENCE LISTING

The contents of the electronic sequence listing (702581.02235.xml; Size: 19,640 bytes; and Date of Creation: Nov. 9, 2022) is herein incorporated by reference in its entirety.

### FIELD OF INVENTION

The field of the invention relates to small molecule inhibitors targeting the sodium channel tamoxifen receptor and the use thereof in treating diseases and disorders associated with voltage-gated sodium channels. In particular, the invention relates to the use of tamoxifen derivatives for the inhibition of sodium channel activity in sensory neurons.

### BACKGROUND AND SUMMARY

Chronic and acute nociceptive pain is complex and often poorly understood, and yet affects more than 50 million patients every year¹³. Current standard of care medications to treat moderate to severe nociceptive pain includes opioids, adjuvants (e.g. non-steroidal anti-inflammatory drugs, NSAIDs) and psychotropic medications (e.g. tricyclic anti-depressants, TCAs)¹⁴. Opioids are effective analgesics, but their use is marred by increased drug tolerance and high risk of developing substance addiction. In addition, commonly used NSAIDs and TCAs have limited efficacy and are associated with an array of serious cardiovascular and cognitive side effects, respectively^{15,16} Several sodium channel inhibitors, such as carbamazepine and cannabidiol, are also used for chronic neuropathic pain conditions (e.g. painful diabetic neuropathy). Given that voltage-gated sodium channel (Naᵥ) inhibition is non-addictive, and the poor efficacy and safety risks associated with psychotropic drugs, targeting peripheral nerve sodium channels for development of future pain medications has garnered considerable pharmaceutical investment¹⁷.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Tamoxifen metabolites inhibit endogenous voltage-gated sodium channels expressed in murine DRG neurons. A) DIC and fluorescence images of voltage clamped DRG neuron isolated and cultured from an Naᵥ1.8-tomato mouse. Continuity with the patched neuron is indicted by the GFP (Alexa Fluor 488 dye 1 nM) loaded into the glass patch electrode. B) Top, exemplar sodium currents (I_{Na}) recorded in control saline and two concentrations of N-desmethyl tamoxifen (ND-Tam) from a voltage-clamped DRG neuron. I_{Na} was activated by 0.2 Hz train of 50 ms depolarizations to −10 mV from −100 mV holding potential. Bottom, time course of peak I_{Na} inhibition during control conditions and after 5 minutes of extracellular ND-Tam treatment. C) Resulting drug concentration-I_{Na} inhibition relationship for ND-Tam fit to the Hill equation. Open symbols represent responses from individual cells and filled symbols represent average response per concentration. Error is equal to S.E.M. (n=11) D) A comparison of the percent I_{Na} recovery after 3-10 μM ND-Tam or 1 mM carbamazepine (CBZ) inhibition. I_{Na} recovery was assessed after 5 minutes in control saline post drug exposure. Fewer I_{Na} recovered from ND-Tam exposure compared to CBZ exposure, 14.3±5.4% and 47.2±9.6%, respectively, and was statistically significant (P=0.03). Error is equal to S.E.M. and the number of cells tested per treatment group is indicated within the parentheses.

FIG. 2. The NavMs tamoxifen receptor site is conserved in domain III and IV of human Naᵥ1.7. A) Structural alignment of human Naᵥ1.7 (PDB: 6J8J) and the 4OH-tamoxifen bound prokaryotic NavMs channel (PDB: 6SXG). Inner (Siteᵢₙ) and outer (Siteₒᵤₜ) 4OH-tam binding sites are indicated. B) Comparison of the tamoxifen receptor site in NavMs, and proposed location in sixth transmembrane segment (S6) Naᵥ1.7 domain III and domain IV. C) Alignments of the S6 in the homotetrameric sodium channel NavMs and in domains I-IV of sensory neuron Naᵥ subtypes using the standard clustal color scheme by amino acid character. NavMs is SEQ ID NO: 1, Nav1.1 domain I is SEQ ID NO: 2, Nav1.1 domain II is SEQ ID NO: 3, Nav1.1 domain III is SEQ ID NO: 4, Nav1.1 domain IV is SEQ ID NO: 5, Nav1.6 domain I is SEQ ID NO: 6, Nav1.6 domain II is SEQ ID NO: 7, Nav1.6 domain III is SEQ ID NO: 8, Nav1.6 domain IV is SEQ ID NO: 9, Nav1.7 domain I is SEQ ID NO: 10, Nav1.7 domain II is SEQ ID NO: 11, Nav1.7 domain III is SEQ ID NO: 12, Nav1.7 domain IV is SEQ ID NO: 13, Nav1.8 domain I is SEQ ID NO: 14, Nav1.8 domain II is SEQ ID NO: 15, Nav1.8 domain III is SEQ ID NO: 16, Nav1.8 domain IV is SEQ ID NO: 17, Nav1.9 domain I is SEQ ID NO: 18, Nav1.9 domain II is SEQ ID NO: 19, Nav1.9 domain III is SEQ ID NO: 20, Nav1.9 domain IV is SEQ ID NO: 21 D) Alanine substitutions to the proposed receptor site in Nav1.7 DIII (D1458A) and DIV (E1761A) reduces ND-Tam potency. E) Drug concentration-I_{Na} inhibition relationship for ND-Tam were fit to the Hill equation. Open symbols represent responses from individual cells and filled symbols represent average response per concentration. Error is equal to S.E.M. and the number of cells tested per treatment group is indicated within the parentheses.

FIG. 3. The mechanism of N-desmethyl tamoxifen inhibition of sensory neuron sodium currents. A) Voltage step protocol (gray) and resulting DRG I_{Na} traces before and after ND-Tam application. B) The impact of ND-Tam on voltage-dependent sodium channel function. Left, conductance (G)-voltage and inactivation-voltage relationships were measured by plotting the average conductance and reduction of test pulse I_{Na}, respectively, as a function of pre-pulse potential. Both relationships were fit to a Boltzmann equation. Open symbols represent responses from individual cells and filled symbols represent average responses. The half-maximal conductance-voltage relationship was not different (P=0.90) when measured in control saline (V_{1/2}=−38.3±0.3 mV) compared to ND-Tam conditions (V_{1/2}=−37.7±0.4 mV). Right, the test pulse current from control and drug conditions normalized to the same scale. The voltage-dependence of inactivation in control conditions and after drug treatment were equal to −47.6±0.4 mV and −51.9±0.4 mV, respectively, and were statistically different (P=0.01), based on results from a paired two-tailed Student's t-test. C) DRG neuron I_{Na} recovery from inactivation (τ_{rec.}) before and after ND-Tam treatment. Sodium currents were inactivated by a 4 ms pre-pulse to 0 mV followed by an identical test pulse separated by increasing recovery times. The ratio of test pulse and pre-pulse current is plotted as a function of recovery time and fit to a single exponential equation. Recovery of I_{Na} from inactivation was delayed after 3 μM ND-Tam exposure (τ_{rec.}=1.1±0.1 ms) compared to control conditions (τ_{rec.}=1.4±0.1 ms) and was statistically significant (P=0.01). Open symbols represent responses from individual cells and filled symbols represent average responses. Error is equal to S.E.M. and the number of cells tested per treatment group is indicated within the parentheses. D) Voltage-dependence of DRG I_{Na} inhibition by ND-Tam. The potency of ND-Tam inhibition of I_{Na} was assessed using the voltage protocol described in FIG. 1B, while using −40 mV, −60 mV and −100 mV holding potentials. The resulting drug concentration-I_{Na} relationships were fit to the Hill equation. The corresponding half maximal inhibitory concentration for each holding potential is listed in FIG. 12 and the P-value from a two-tailed Student's t-test comparing −100 mV to −60 mV and −40 mV results are indicated on the graph. Open symbols represent responses from individual cells and filled symbols represent average responses. Error is equal to S.E.M. and the number of cells tested per treatment group is indicated within the parentheses.

FIG. 4. N-desmethyl tamoxifen inhibits DRG neuronal transmission. A) Exemplar action potentials recorded from an isolated DRG neuron in control and ND-Tam-treated conditions using a 1 second, 80 pA injection of current. After the experiment was complete, drug was washed out of the bath for five minutes and recovery from inhibition was retested. Consistent with previous reports, the resting membrane potential of the DRG neurons was −53.4±5 mV (Error is equal to S.D., n=109) (PMID: 8052416). B, C) The potency of ND-Tam inhibition on DRG action potential frequency and peak amplitude. Open symbols represent responses from individual cells while filled symbols represent average response per concentration. Error is equal to S.E.M. and the number of cells tested per treatment group is indicated within the parentheses. D, E) A comparison of the potency of cannabidiol (CBD) and carbamazepine (CBZ) inhibition on action potential frequency and peak amplitude. As done previously, drug concentration-I_{Na} inhibition relationships were fit to the Hill equation. The corresponding half maximal inhibitory concentration for each drug is listed in FIG. 14. Open symbols represent responses from individual cells while filled symbols represent average response per concentration. Error is equal to S.E.M. and the number of cells tested per treatment group is indicated within the parentheses.

FIG. 5. ND-Tam produces analgesia in mice with knee hyperalgesia post DMM surgery. A) Histology of paraformaldehyde-fixed murine knee joint, confirming the presence of Nav1.8-tdTomato positive sensory neurons immunostained with anti-sodium channel (Sigma, S8809) and anti-ChAT antibodies (Invitrogen, PA5-29653). B) Higher magnification of the synovial knee joint indicating the locations of the tibia (Tb), medial meniscus (Mm) and patella (Pat). C) Knee withdrawal threshold was measured before intra-articular injection of vehicle (50% EtOH) or 50 μM ND-Tam and then at 30 min, 1 h, 2 h, and 24 h post-injection. ND-Tam provided relief from hyperalgesia compared to vehicle 30 minutes after injection (n=6, P=0.008) D) Knee withdrawal thresholds assessed before and after intra-articular injection of vehicle (0.1% DMSO) and 50 μM ND-Tam, 50 μM CBD, or 50 μM Lidocaine at 30 min, 1 h, 2 h, 3 h, and 24 h time points. P-values are indicated on the graph for time points at which all three drugs are significantly different from vehicle. E) Area under the curve analysis of withdrawal thresholds for vehicle, ND-Tam, CBD, and Lidocaine from 0-3 hours after injection. ND-Tam performs similar to CBD and to lidocaine over time (n=5, P=0.17, P=0.13, respectively). Two-way repeated measures ANOVA were performed on each dataset and resulting P-values for all comparisons are listed in FIG. 15.

FIG. 6. The potency of tamoxifen metabolites against the sodium current conducted by human sensory neuron Naᵥ subtypes. A) Exemplar sodium currents before and after inhibition by N-desmethyl tamoxifen. I_{Na} recorded from cells heterologously expressing human Naᵥs in control and drug-treated conditions. hNaᵥ1.1, hNaᵥ1.6, and hNaᵥ1.7 sodium currents were recorded from transiently transfected HEK cells. I_{Na} conducted by the Naᵥ1.8 subtype was recorded from stably expressed CHO cells. B) The potency of N-desmethyl tamoxifen (left) and 4-hydroxytamoxifen (right) as assessed by fitting the average concentration-I_{Na} inhibition relationships to the Hill equation. The corresponding half maximal inhibitory concentration is listed in FIG. 12. Filled symbols represent average response per concentration. Number of cells tested per Naᵥ subtype and treatment group are indicated within the parenthesis. Error is equal to S.E.M.

FIG. 7. Structure related activity of analogs at the Naᵥ tamoxifen receptor, as assessed by competitive binding results and DRG sodium current inhibition. A) Molecular structures of the tested tamoxifen analogues. B) Normalized sodium current inhibition recorded DRG responses (black) and percent specific binding of tritium labeled tamoxifen (red) to membrane preparations expressing Naᵥ1.7, plotted as a function of tamoxifen analog concentration. Open symbols are results recorded from individual neurons. Filled symbols represent the average response at each concentration. Error is equal to S.E.M.

FIG. 8. Proposed ‘bind and plug’ mechanism by sodium channel inhibition by tamoxifen analogues. Structural alignments of the tamoxifen-bound NavMs (PDB: 6SXG) and with Naᵥ1.7 (PDB: 6J8J), as described in FIG. 2. Narrowest distance between DII (S989) and DIV (E1761) bound analogs along Na⁺ conducting pathway are indicated by the dotted line.

FIG. 9. Voltage-dependent inhibition of sodium currents by N-desmethyl tamoxifen, carbamazepine, cannabidiol and lidocaine. The potency of drug inhibition of I_{Na} was assessed using the voltage protocol described in FIG. 1B, while using −60 mV and −100 mV holding potentials. The drug concentration-I_{Na} relationships were fit to the Hill equation. The corresponding half maximal inhibitory concentration for each holding potential is listed in FIG. 12 and the P-value from a two-tailed Student's t-test comparing −100 mV to −60 mV results are indicated on the graph. Open symbols represent responses from individual cells and filled symbols represent average response per concentration. Error is equal to S.E.M. and the number of cells tested per treatment group is indicated within the parentheses.

FIG. 10. N-desmethyl tamoxifen inhibits spontaneous action potentials in sensory neurons. A) Exemplar spontaneous action potentials, recorded from a DRG neuron without injecting current, inhibited by 100 nM N-desmethyl tamoxifen. B) Morphology of a single spontaneous action potential in DRG neuron C) Inhibition of spontaneous action potential frequency by 100 nM ND-Tam (P=0.04). Open symbols represent responses from individual cells and filled symbols represent the average response. Error is equal to S.E.M. from 6 spontaneously firing DRG neurons.

FIG. 11. Naᵥ1.8-positive sensory neuron in the murine synovial knee joint. A) 10× and B) 60× magnification images of immunostained, paraformaldehyde-fixed, sections of a Nav1.8-tomato mouse knee joints. The locations of the tibia (Tb), medial meniscus (Mm) and femur (Fem) are indicated. Primary antibodies raised against all Naᵥ subtypes, anti-NeuN antibody, and cell membrane stain.

FIG. 12. Drug potency of I_{Na} inhibition from endogenous and heterologous sources. Half maximal inhibitory concentration (IC₅₀) are listed, with error (S.E.M.) and number of cells tested within the parenthesis.

FIG. 13. Drug potency of DRG neuronal transmission. Half maximal inhibitory concentration (IC₅₀) are listed, with error (S.E.M.) and number of cells tested within the parenthesis.

FIG. 14. Drug potency of DRG neuronal transmission inhibition. Half maximal inhibitory concentrations (IC50) are listed, with error (S.E.M.) and number of cells tested within the parenthesis.

FIG. 15. Statistics comparing ND-Tam, CBD, and Lidocaine in the treatment of knee hyperalgesia A) Resulting P-values from two-way repeated measures ANOVA with Sidak's multiple comparisons test for FIG. 5A. B) Resulting P-values from two-way repeated measures ANOVA with Dunnett's multiple comparisons test for FIG. 5B. C) Resulting P-values from two-way repeated measures ANOVA for FIG. 5C.

FIG. 16. Shows potential novel tamoxifen analogues of the current disclosure.

### DETAILED DESCRIPTION

The following disclosure provides compositions and methods for treating nociceptive pain. The inventors made the surprising discovery that tamoxifen, and derivatives thereof, bind to a previously undescribed location in voltage gated sodium channels (Naᵥs). Therefore, the inventors demonstrate that derivatives with poor estrogen receptor antagonism, for example N-desmethyl tamoxifen, are effective at antagonizing Naᵥs. Furthermore, the inventors demonstrate additional novel compounds that have excellent specificity for Naᵥs.

The present invention is described herein using several definitions, as set forth below and throughout the application.

### Definitions

As used herein the term “analgesic” may be defined as a drug designed to control pain.

As used herein the term “chronic pain” may be defined as pain that persists for a period of time (e.g., greater than three months). In contrast to acute pain, chronic pain can continue after the injury or illness that caused it has healed or been resolved. Chronic pain may also occur in some subjects in the absence of apparent injury or illness.

As used herein the term “hyperexcitability” may be defined as a state of a neuron, or network of neurons, in which the likelihood that the neuron will be activated (depolarize) is increased. This may be due to the aberrant expression patterns of Naᵥs after traumatic injury and inflammation. Hyperexcitability may lead to conditions such as chronic pain. Neurons that exhibit the state of hyperexcitability are said to be hyperexcitable.

As used herein the term “membrane depolarization” may be defined as a process of changing the electrical potential across a biological membrane. More typically, membrane depolarization refers to the process of neuronal depolarization wherein voltage-gated ion channels open on the surface of the neuronal membrane that rapidly change the electrical potential across the membrane of the neuron from about highly negative (−70 mV) to about zero (0 mV) or about slightly positive (+30 mV).

As used herein the term “nociceptive pain” may be defined as pain that is caused by the interpretation of signals sent by nociceptive receptors that sense potentially harmful stimuli.

As used herein the term “neuropathic pain” may be defined as pain caused by the damage to neurons or cells of the nervous system. Non-limiting examples of neuropathic pain include pain secondary to diabetic neuropathy or multiple sclerosis (MS).

As used herein the term “state-dependent accessibility” may be defined as a state in which voltage-gated sodium channels (Naᵥs) undergo changes in structural conformation when neuronal membranes are depolarized, transitioning from closed, to open, to inactivated, and back to closed during the course of membrane depolarization. Therefore, if the ability of a molecule to enter a specific site on a voltage-gated sodium channel depends on the state of the channel, i.e. closed, open, or inactivated, then the molecule displays state-dependent accessibility. This property is desirable in the context of design of analgesic drugs because it confers additional specificity to the drug action by, for example, only targeting voltage-gated sodium channels present on neurons that are in the process of depolarizing and are in the open conformation. This restricts drug action to aberrantly firing neurons, or neurons contributing to pain, and spares other neurons from inhibition.

As used herein the term “voltage-gated sodium channel” or Naᵥs may be defined as integral membrane proteins that form ion channels and allow the passage of Na⁺ ions through a cell's plasma membrane in response to the increase of cellular membrane potential from about −70 mV to about −55 mV. In vertebrates, there are nine Naᵥs: Naᵥ1.1-1.9. In mice and humans, Naᵥ1.1, Naᵥ1.6, Naᵥ1.7, Naᵥ1.8, and Naᵥ1.9 are all expressed by adult sensory neurons. Naᵥs exist in three distinct conformational states: resting, open, and inactivated. Of these three conformational states, only open channels have the ability to conduct Na⁺ ions across the cell membrane.

The disclosed subject matter may be further described using definitions and terminology as follows. The definitions and terminology used herein are for the purpose of describing particular embodiments only and are not intended to be limiting.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

A “subject in need thereof” as utilized herein may refer to a subject in need of treatment for a disease or disorder associated with hyperexcitability of sensory neurons and/or expression or activity of voltage-gated sodium channels. A subject in need thereof may include a subject having nociceptive pain, inflammatory pain, neuropathic pain, chronic pain, or pain associated with osteoarthritis. A subject in need thereof may also include a subject having chronic or chronic nociceptive pain.

The term “subject” may be used interchangeably with the terms “individual” and “patient” and includes human and non-human mammalian subjects.

The disclosed compounds, pharmaceutical compositions, and methods may be utilized to treat diseases and disorders associated with voltage-gated sodium channel activity and/or expression which may include, but are not limited to pain, chronic pain, chronic nociceptive pain. The disclosed compounds may be utilized to modulate the biological activity of Naᵥ1.1, Naᵥ1.6, Naᵥ1.7, Naᵥ1.8, and Naᵥ1.9. The term “modulate” should be interpreted broadly to include “inhibiting” voltage-gated sodium channel biological activity including voltage-gated ion channel activity.

Chemical Entities

Chemical entities and the use thereof may be disclosed herein and may be described using terms known in the art and defined herein.

The term “alkyl” as used herein refers to a saturated straight or branched hydrocarbon, such as a straight or branched group of 1-12, 1-10, or 1-6 carbon atoms, referred to herein as C₁-C₁₂ alkyl, C₁-C₁₀-alkyl, and C₁-C₆-alkyl, respectively.

The term “alkylene” refers to a diradical of an alkyl group. An exemplary alkylene group is —CH₂CH₂—.

The term “haloalkyl” refers to an alkyl group that is substituted with at least one halogen, for example, —CH₂F, —CHF₂, —CF₃, —CH₂CF₃, —CF₂CF₃, and the like.

The term “heteroalkyl” as used herein refers to an “alkyl” group in which at least one carbon atom has been replaced with a heteroatom (e.g., an O, N, or S atom). One type of heteroalkyl group is an “alkoxyl” group.

The term “alkenyl” as used herein refers to an unsaturated straight or branched hydrocarbon having at least one carbon-carbon double bond, such as a straight or branched group of 2-12, 2-10, or 2-6 carbon atoms, referred to herein as C₂-C₁₂-alkenyl, C₂-C₁₀-alkenyl, and C₂-C₆-alkenyl, respectively. A “cycloalkene” is a compound having a ring structure (e.g., of 3 or more carbon atoms) and comprising at least one double bond.

The term “alkynyl” as used herein refers to an unsaturated straight or branched hydrocarbon having at least one carbon-carbon triple bond, such as a straight or branched group of 2-12, 2-10, or 2-6 carbon atoms, referred to herein as C₂-C₁₂-alkynyl, C₂-C₁₀-alkynyl, and C₂-C₆-alkynyl, respectively.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₄₋₈-cycloalkyl,” derived from a cycloalkane. Unless specified otherwise, cycloalkyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the cycloalkyl group is not substituted, i.e., it is unsubstituted.

The term “cycloalkylene” refers to a diradical of a cycloalkyl group.

The term “partially unsaturated carbocyclyl” refers to a monovalent cyclic hydrocarbon that contains at least one double bond between ring atoms where at least one ring of the carbocyclyl is not aromatic. The partially unsaturated carbocyclyl may be characterized according to the number or ring carbon atoms. For example, the partially unsaturated carbocyclyl may contain 5-14, 5-12, 5-8, or 5-6 ring carbon atoms, and accordingly be referred to as a 5-14, 5-12, 5-8, or 5-6 membered partially unsaturated carbocyclyl, respectively. The partially unsaturated carbocyclyl may be in the form of a monocyclic carbocycle, bicyclic carbocycle, tricyclic carbocycle, bridged carbocycle, spirocyclic carbocycle, or other carbocyclic ring system. Exemplary partially unsaturated carbocyclyl groups include cycloalkenyl groups and bicyclic carbocyclyl groups that are partially unsaturated. Unless specified otherwise, partially unsaturated carbocyclyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the partially unsaturated carbocyclyl is not substituted, i.e., it is unsubstituted.

The term “aryl” is art-recognized and refers to a carbocyclic aromatic group. Representative aryl groups include phenyl, naphthyl, anthracenyl, and the like. The term “aryl” includes polycyclic ring systems having two or more carbocyclic rings in which two or more carbons are common to two adjoining rings (the rings are “fused rings”) wherein at least one of the rings is aromatic and, e.g., the other ring(s) may be cycloalkyls, cycloalkenyls, cycloalkynyls, and/or aryls. Unless specified otherwise, the aromatic ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. In certain embodiments, the aromatic ring is substituted at one or more ring positions with halogen, alkyl, hydroxyl, or alkoxyl. In certain other embodiments, the aromatic ring is not substituted, i.e., it is unsubstituted. In certain embodiments, the aryl group is a 6-10 membered ring structure.

The terms “heterocyclyl” and “heterocyclic group” are art-recognized and refer to saturated, partially unsaturated, or aromatic 3- to 10-membered ring structures, alternatively 3- to 7-membered rings, whose ring structures include one to four heteroatoms, such as nitrogen, oxygen, and sulfur. The number of ring atoms in the heterocyclyl group can be specified using 5 Cx-Cx nomenclature where x is an integer specifying the number of ring atoms. For example, a C₃-C₇ heterocyclyl group refers to a saturated or partially unsaturated 3- to 7-membered ring structure containing one to four heteroatoms, such as nitrogen, oxygen, and sulfur. The designation “C₃-C₇” indicates that the heterocyclic ring contains a total of from 3 to 7 ring atoms, inclusive of any heteroatoms that occupy a ring atom position.

The terms “amine” and “amino” are art-recognized and refer to both unsubstituted and substituted amines, wherein substituents may include, for example, alkyl, cycloalkyl, heterocyclyl, alkenyl, and aryl.

The terms “alkoxyl” or “alkoxy” are art-recognized and refer to an alkyl group, as defined above, having an oxygen radical attached thereto. Representative alkoxyl groups include methoxy, ethoxy, tert-butoxy and the like.

An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, and the like.

The term “carbonyl” as used herein refers to the radical —C(O)—.

The term “carboxy” or “carboxyl” as used herein refers to the radical —COOH or its corresponding salts, e.g. —COONa, etc.

The term “amide” or “amido” or “carboxamido” as used herein refers to a radical of the form —R¹C(O)N(R²)—, —R¹C(O)N(R²)R³—, —C(O)NR²R³, or —C(O)NH₂, wherein R¹, R² and R³ are each independently alkoxy, alkyl, alkenyl, alkynyl, amide, amino, aryl, arylalkyl, carbamate, cycloalkyl, ester, ether, formyl, halogen, haloalkyl, heteroaryl, heterocyclyl, hydrogen, hydroxyl, ketone, or nitro.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present invention encompasses various stereo isomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly. It is understood that graphical depictions of chemical structures, e.g., generic chemical structures, encompass all stereoisomeric forms of the specified compounds, unless indicated otherwise.

Pharmaceutical Compositions

The compounds employed in the compositions and methods disclosed herein may be administered as pharmaceutical compositions and, therefore, pharmaceutical compositions incorporating the compounds are considered to be embodiments of the compositions disclosed herein. Such compositions may take any physical form which is pharmaceutically acceptable; illustratively, they can be orally administered pharmaceutical compositions. Such pharmaceutical compositions contain an effective amount of a disclosed compound, which effective amount is related to the daily dose of the compound to be administered. Each dosage unit may contain the daily dose of a given compound or each dosage unit may contain a fraction of the daily dose, such as one-half or one-third of the dose. The amount of each compound to be contained in each dosage unit can depend, in part, on the identity of the particular compound chosen for the therapy and other factors, such as the indication for which it is given. The pharmaceutical compositions disclosed herein may be formulated so as to provide quick, sustained, or delayed release of the active ingredient after administration to the patient by employing well known procedures.

The compounds for use according to the methods of disclosed herein may be administered as a single compound or a combination of compounds. For example, a compound that activates the biological activity of voltage-gated sodium channels may be administered as a single compound or in combination with another compound that activates the biological activity of voltage-gated sodium channels or that has a different pharmacological activity.

As indicated above, pharmaceutically acceptable salts of the compounds are contemplated and also may be utilized in the disclosed methods. The term “pharmaceutically acceptable salt” as used herein, refers to salts of the compounds, which are substantially non-toxic to living organisms. Typical pharmaceutically acceptable salts include those salts prepared by reaction of the compounds as disclosed herein with a pharmaceutically acceptable mineral or organic acid or an organic or inorganic base. Such salts are known as acid addition and base addition salts. It will be appreciated by the skilled reader that most or all of the compounds as disclosed herein are capable of forming salts and that the salt forms of pharmaceuticals are commonly used, often because they are more readily crystallized and purified than are the free acids or bases.

Acids commonly employed to form acid addition salts may include inorganic acids such as hydrochloric acid, hydrobromic acid, hydroiodic acid, sulfuric acid, phosphoric acid, and the like, and organic acids such as p-toluenesulfonic, methanesulfonic acid, oxalic acid, p-bromophenylsulfonic acid, carbonic acid, succinic acid, citric acid, benzoic acid, acetic acid, and the like. Examples of suitable pharmaceutically acceptable salts may include the sulfate, pyrosulfate, bisulfate, sulfite, bisulfate, phosphate, monohydrogenphosphate, dihydrogenphosphate, metaphosphate, pyrophosphate, bromide, iodide, acetate, propionate, decanoate, caprylate, acrylate, formate, hydrochloride, dihydrochloride, isobutyrate, caproate, heptanoate, propiolate, oxalate, malonate, succinate, suberate, sebacate, fumarate, maleat-, butyne-.1,4-dioate, hexyne-1,6-dioate, benzoate, chlorobenzoate, methylbenzoate, hydroxybenzoate, methoxybenzoate, phthalate, xylenesulfonate, phenylacetate, phenylpropionate, phenylbutyrate, citrate, lactate, α-hydroxybutyrate, glycolate, tartrate, methanesulfonate, propanesulfonate, naphthalene-1-sulfonate, naphthalene-2-sulfonate, mandelate, and the like.

Base addition salts include those derived from inorganic bases, such as ammonium or alkali or alkaline earth metal hydroxides, carbonates, bicarbonates, and the like. Bases useful in preparing such salts include sodium hydroxide, potassium hydroxide, ammonium hydroxide, potassium carbonate, sodium carbonate, sodium bicarbonate, potassium bicarbonate, calcium hydroxide, calcium carbonate, and the like.

The particular counter-ion forming a part of any salt of a compound disclosed herein is may not be critical to the activity of the compound, so long as the salt as a whole is pharmacologically acceptable and as long as the counter-ion does not contribute undesired qualities to the salt as a whole. Undesired qualities may include undesirably solubility or toxicity.

Pharmaceutically acceptable esters and amides of the compounds can also be employed in the compositions and methods disclosed herein. Examples of suitable esters include alkyl, aryl, and aralkyl esters, such as methyl esters, ethyl esters, propyl esters, dodecyl esters, benzyl esters, and the like. Examples of suitable amides include unsubstituted amides, monosubstituted amides, and disubstituted amides, such as methyl amide, dimethyl amide, methyl ethyl amide, and the like.

In addition, the methods disclosed herein may be practiced using solvate forms of the compounds or salts, esters, and/or amides, thereof. Solvate forms may include ethanol solvates, hydrates, and the like.

The pharmaceutical compositions may be utilized in methods of treating a disease or disorder associated with the biological activity of voltage-gated sodium channels. As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder. As such, the methods disclosed herein encompass both therapeutic and prophylactic administration.

As used herein the term “effective amount” refers to the amount or dose of the compound, upon single or multiple dose administration to the subject, which provides the desired effect in the subject under diagnosis or treatment. The disclosed methods may include administering an effective amount of the disclosed compounds (e.g., as present in a pharmaceutical composition) for treating a disease or disorder associated with biological activity of voltage-gated sodium channels. The disclosed methods may include administering an effective amount of the disclosed compounds for treating a nociceptive pain, inflammatory pain, neuropathic pain, chronic pain, or pain associated with osteoarthritis. The disclosed methods may also include administering an effective amount of the disclosed compounds for treating chronic or chronic nociceptive pain.

An effective amount can be readily determined by the attending diagnostician, as one skilled in the art, by the use of known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances.

A typical daily dose may contain from about 0.01 mg/kg to about 100 mg/kg (such as from about 0.05 mg/kg to about 50 mg/kg and/or from about 0.1 mg/kg to about 25 mg/kg) of each compound used in the present method of treatment.

Compositions can be formulated in a unit dosage form, each dosage containing from about 1 to about 500 mg of each compound individually or in a single unit dosage form, such as from about 5 to about 300 mg, from about 10 to about 100 mg, and/or about 25 mg. The term “unit dosage form” refers to a physically discrete unit suitable as unitary dosages for a patient, each unit containing a predetermined quantity of active material calculated to produce the desired therapeutic effect, in association with a suitable pharmaceutical carrier, diluent, or excipient.

Oral administration is an illustrative route of administering the compounds employed in the compositions and methods disclosed herein. Other illustrative routes of administration include transdermal, percutaneous, intravenous, intramuscular, intranasal, buccal, intrathecal, intracerebral, or intrarectal routes. The route of administration may be varied in any way, limited by the physical properties of the compounds being employed and the convenience of the subject and the caregiver.

As one skilled in the art will appreciate, suitable formulations include those that are suitable for more than one route of administration. For example, the formulation can be one that is suitable for both intrathecal and intracerebral administration. Alternatively, suitable formulations include those that are suitable for only one route of administration as well as those that are suitable for one or more routes of administration, but not suitable for one or more other routes of administration. For example, the formulation can be one that is suitable for oral, transdermal, percutaneous, intravenous, intramuscular, intranasal, buccal, and/or intrathecal administration but not suitable for intracerebral administration.

The inert ingredients and manner of formulation of the pharmaceutical compositions are conventional. The usual methods of formulation used in pharmaceutical science may be used here. All of the usual types of compositions may be used, including tablets, chewable tablets, capsules, solutions, parenteral solutions, intranasal sprays or powders, troches, suppositories, transdermal patches, and suspensions. In general, compositions contain from about 0.5% to about 50% of the compound in total, depending on the desired doses and the type of composition to be used. The amount of the compound, however, is best defined as the “effective amount”, that is, the amount of the compound which provides the desired dose to the patient in need of such treatment. The activity of the compounds employed in the compositions and methods disclosed herein are not believed to depend greatly on the nature of the composition, and, therefore, the compositions can be chosen and formulated primarily or solely for convenience and economy.

Capsules are prepared by mixing the compound with a suitable diluent and filling the proper amount of the mixture in capsules. The usual diluents include inert powdered substances (such as starches), powdered cellulose (especially crystalline and microcrystalline cellulose), sugars (such as fructose, mannitol and sucrose), grain flours, and similar edible powders.

Tablets are prepared by direct compression, by wet granulation, or by dry granulation. Their formulations usually incorporate diluents, binders, lubricants, and disintegrators (in addition to the compounds). Typical diluents include, for example, various types of starch, lactose, mannitol, kaolin, calcium phosphate or sulfate, inorganic salts (such as sodium chloride), and powdered sugar. Powdered cellulose derivatives can also be used. Typical tablet binders include substances such as starch, gelatin, and sugars (e.g., lactose, fructose, glucose, and the like). Natural and synthetic gums can also be used, including acacia, alginates, methylcellulose, polyvinylpyrrolidine, and the like. Polyethylene glycol, ethylcellulose, and waxes can also serve as binders.

Tablets can be coated with sugar, e.g., as a flavor enhancer and sealant. The compounds also may be formulated as chewable tablets, by using large amounts of pleasant-tasting substances, such as mannitol, in the formulation. Instantly dissolving tablet-like formulations can also be employed, for example, to assure that the patient consumes the dosage form and to avoid the difficulty that some patients experience in swallowing solid objects.

A lubricant can be used in the tablet formulation to prevent the tablet and punches from sticking in the die. The lubricant can be chosen from such slippery solids as talc, magnesium and calcium stearate, stearic acid, and hydrogenated vegetable oils.

Tablets can also contain disintegrators. Disintegrators are substances that swell when wetted to break up the tablet and release the compound. They include starches, clays, celluloses, algins, and gums. As further illustration, corn and potato starches, methylcellulose, agar, bentonite, wood cellulose, powdered natural sponge, cation-exchange resins, alginic acid, guar gum, citrus pulp, sodium lauryl sulfate, and carboxymethylcellulose can be used.

Compositions can be formulated as enteric formulations, for example, to protect the active ingredient from the strongly acid contents of the stomach. Such formulations can be created by coating a solid dosage form with a film of a polymer which is insoluble in acid environments and soluble in basic environments. Illustrative films include cellulose acetate phthalate, polyvinyl acetate phthalate, hydroxypropyl methylcellulose phthalate, and hydroxypropyl methylcellulose acetate succinate.

Transdermal patches can also be used to deliver the compounds. Transdermal patches can include a resinous composition in which the compound will dissolve or partially dissolve; and a film which protects the composition, and which holds the resinous composition in contact with the skin. Other, more complicated patch compositions can also be used, such as those having a membrane pierced with a plurality of pores through which the drugs are pumped by osmotic action.

As one skilled in the art will also appreciate, the formulation can be prepared with materials (e.g., actives excipients, carriers (such as cyclodextrins), diluents, etc.) having properties (e.g., purity) that render the formulation suitable for administration to humans. Alternatively, the formulation can be prepared with materials having purity and/or other properties that render the formulation suitable for administration to non-human subjects, but not suitable for administration to humans.

Inhibitors of Voltage-Gated Sodium Channels and Uses Thereof

The disclosed methods may include treating pain in a subject in need thereof, the method comprising administering to the subject an effective amount of a compound having a formula as follows or a suitable pharmaceutical salt thereof:

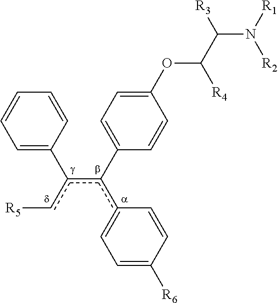

wherein:
R¹ is selected from hydrogen, alkyl, alkoxy, hydroxy, and halogen;
R² is selected from hydrogen, alkyl, alkoxy, hydroxy, and halogen;
R³ is selected from hydrogen, alkyl, alkoxy, hydroxy, halogen, and amino;
R⁴ is selected from hydrogen, alkyl, alkoxy, hydroxy, halogen, and amino;
R⁵ is selected from hydrogen, alkyl, alkoxy, hydroxy, halogen, and amino; and
R⁶ is selected from hydrogen and hydroxy when there are single bonds between C^{α}-C^{β} and C^{γ}-C^{δ} and a double bond between C^{β}-C^{γ} or R⁶ is oxo when there are double bonds between C^{α}-C^{β} and C^{γ}-C^{δ} and a single bond between C^{β}-C^{γ}.

Exemplary R¹⁻⁵ alkyl groups include, without limitation, methyl or ethyl.

Exemplary R¹⁻⁵ alkoxy groups include, without limitation, methoxy.

Exemplary R¹⁻⁵ halogen groups include, without limitation, chloro and bromo.

Exemplary R³⁻⁵ amino groups include, without limitation, —NH₂.

In some embodiments, R¹⁻² may be independently selected from hydrogen and methyl, R³ is hydrogen, and R⁴ is hydrogen.

In some embodiments the compound is tamoxifen (2-[4-[(Z)-1,2-diphenylbut-1-enyl]phenoxy]-N,N-dimethylethanamine) or a metabolite thereof, e.g., N-desmethyltamoxifen (ND-Tam; 2-[4-[(Z)-1,2-diphenylbut-1-enyl]phenoxy]-N-methylethanamine), 4-hydroxytamoxifen (4OH-Tam; 4-[(Z)-1-[4-[2-(dimethylamino)ethoxy]phenyl]-2-phenylbut-1-enyl]phenol), N-desmethyl-4-hydroxytamoxifen (endoxifen; 4-[(Z)-1-[4-[2-(methylamino)ethoxy]phenyl]-2-phenylbut-1-enyl]phenol), or 4-hydroxytamoxifen quinone methide ((4-[(E)-1-[4-[2-(dimethylamino)ethoxy]phenyl]-2-phenylbut-2-enylidene]cyclohexa-2,5-dien-1-one).

In some embodiments the compound is not any of tamoxifen, N-desmethyltamoxifen, 4-hydroxytamoxifen, N-desmethyl-4-hydroxytamoxifen, or 4-hydroxytamoxifen quinone methide.

Tamoxifen is a prodrug that requires metabolism to form pharmacologically active metabolites, such as 4-hydroxytamoxifen or N-desmethyl-4-hydroxytamoxifen (endoxifen) by cytochrome P450 (CYP)-mediate catalysis. These R⁶ hydroxylated metabolites of tamoxifen have high binding affinity for estrogen receptor (ER) and exert antiestrogenic and antitumor activities. Metabolites, such as N-desmethyltamoxifen where R⁶ is hydrogen, have lower binding affinity for ER while possessing half maximal potency of I_{Na} inhibition (IC₅₀) comparable to or superior to R⁶ hydroxylated metabolites. In some embodiments, it may be beneficial to treat a subject with a compound that binds and/or inhibits a Naᵥ but which has low affinity for ER.

The disclosed methods may also include treating pain in a subject in need thereof, the method comprising administering to the subject an effective amount of a compound having a formula as follows or a suitable pharmaceutical salt thereof:

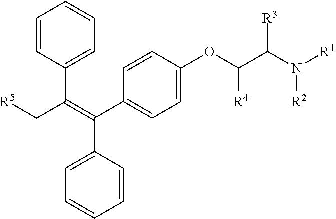

wherein:
R¹ is selected from hydrogen, alkyl, alkoxy, hydroxy, and halogen;
R² is selected from hydrogen, alkyl, alkoxy, hydroxy, and halogen;
R³ is selected from hydrogen, alkyl, alkoxy, hydroxy, halogen, and amino;
R⁴ is selected from hydrogen, alkyl, alkoxy, hydroxy, halogen, and amino; and
R⁵ is selected from hydrogen, alkyl, alkoxy, hydroxy, halogen, and amino.

In some embodiments, the subject has nociceptive pain. In some embodiments, the subject has inflammatory pain. In some embodiments, the subject has neuropathic pain. In some embodiments, the subject has chronic pain. In some embodiments, the subject has pain associated with osteoarthritis. In some embodiments, the subject has pain associated with an activated voltage-gated sodium channel (Naᵥs).

In some embodiments, the disclosed methods of treating pain may involve administering the compound orally. In other embodiments, the disclosed methods of treating pain may involve administering the disclosed compound topically. In some embodiments, the disclosed methods of treating pain may involve administering the disclosed compound intravenously.

In some embodiments, the subject is administered a dose of the compound that is effective for achieving a concentration of the compound at the site of action of at least about 1 nM, 2 nM, 3 nM, 4 nM, 5 nM, 10 nM, 20 nM, 30 nM, 40 nM, 50 nM or higher.

In some embodiments, the subject is administered a daily oral dose of the compound of less than about 300 mg, 200 mg, 100 mg, 50 mg, 40 mg, 30 mg, 20 mg, 10 mg, 5 mg, 4 mg, 3 mg, 2 mg, 1 mg, or less, or dose within a range bounded by any of these values.

In some embodiments, the subject is administered a daily dose of the compound of less than about 10 mg/kg, 5 mg/kg, 1 mg/kg, 0.5 mg/kg, 0.1 mg/kg, 0.05 mg/kg, 0.01 mg/kg or lower, or within a range bounded by any of these values.

In some embodiments, the compound does not bind to the estrogen receptor or the compound binds to the estrogen receptor and has a dissociation constant (K_{d}) for the estrogen receptor which greater than about 100 nM, 200 nM, 300 nM, 400 nM, 500 nM, 600 nM, 700 nM, 800 nM, 900 nM, 1000 nM or higher.

In some embodiments, the compound binds to a voltage-gated sodium channel (Naᵥ) and has a dissociation constant (K_{d}) less than about 50 nM, 40 nM, 30 nM, 20 nM, 10 nM, 9 nM, 8 nM, 7 nM, 6 nM, 5 nM, 4 nM, 3 nM, 2 nM, 1 nM, or lower.

In some embodiments, the compound binds to a voltage-gated sodium channel (Naᵥ) and has a dissociation constant (K_{d}) less than about 50 nM, 40 nM, 30 nM, 20 nM, 10 nM, 9 nM, 8 nM, 7 nM, 6 nM, 5 nM, 4 nM, 3 nM, 2 nM, 1 nM, or lower and the compound does not bind to the estrogen receptor or the compound binds to the estrogen receptor and has a dissociation constant (K_{d}) for the estrogen receptor which greater than about 100 nM, 200 nM, 300 nM, 400 nM, 500 nM, 600 nM, 700 nM, 800 nM, 900 nM, 1000 nM or higher.

In some embodiments, the compound inhibits the activity of a voltage-gated sodium channel (Naᵥ) and has a half-maximal inhibitory concentration (IC₅₀) of less than about 200 nM, 100 nM, 90 nM, 80 nM, 70 nM, 60 nM, 50 nM, 40 nM, 30 nM, 20 nM, 10 nM, or lower.

In some embodiments, the compound inhibits the activity of a voltage-gated sodium channel (Naᵥ) and has a half-maximal inhibitory concentration (IC₅₀) of less than about 200 nM, 100 nM, 90 nM, 80 nM, 70 nM, 60 nM, 50 nM, 40 nM, 30 nM, 20 nM, 10 nM, or lower and the compound does not bind to the estrogen receptor or the compound binds to the estrogen receptor and has a dissociation constant (K_{d}) for the estrogen receptor which greater than about 100 nM, 200 nM, 300 nM, 400 nM, 500 nM, 600 nM, 700 nM, 800 nM, 900 nM, 1000 nM or higher.

In some embodiments, the binds to and/or inhibits a Naᵥ selected from Naᵥ1.1, Naᵥ1.2, Naᵥ1.3, Naᵥ1.4, Naᵥ1.5, Naᵥ1.6, Naᵥ1.7, Naᵥ1.8, and Naᵥ1.9.

### EXAMPLES

The following Examples are illustrative and should not be interpreted to limit the scope of the claimed subject matter.

### Example 1—Targeting the Sodium Channel Tamoxifen Receptor to Block Nociceptive Pain

Voltage-gated sodium channels (Naᵥs) initiate action potentials required for the transmission of painful stimuli by nociceptive neurons. Targeting Na s with drugs to produce analgesic effects for pain management is an active area of research. Recent advances in structural biology tools have enabled the resolution of several subtypes of Na s, providing a blueprint for drug design. The Examples characterize the pharmacology of ND-Tam against eukaryotic Naᵥs natively expressed in sensory neurons. The Examples demonstrate that ND-Tam inhibition of sodium current is state dependent. This feature confers frequency- and voltage-dependent inhibition of action potentials in nociceptive neurons. ND-Tam achieves high nanomolar potencies for inhibition of neuronal transmission from cultured nerves, indicating that this drug acts preferentially against hyperexcitable sensory neurons responsible for aberrant painful stimuli. Based on results from competitive binding experiments, chemical structure-related activity screening and functional mutagenesis analysis, ND-Tam and analogues occupy two conserved intracellular receptor sites in domain II and IV to block Na⁺ conduction using a ‘bind and plug’ strategy. When tested using an osteoarthritis mouse model of pain, ND-Tam had long lasting analgesic effects when applied to the joint in vivo, which is consistent with its apparent slow dissociation from the channel. ND-Tam outperforms clinically used pain medications, carbamazepine and cannabidiol, in blocking sodium current, sensory neuron transmission, and in vivo efficacy using an osteoarthritis pain mouse model. The Examples demonstrate that targeting this Naᵥ receptor with prototypic tamoxifen analogs is a viable strategy for pain management.

Voltage-gated sodium channels (Naᵥs) are transmembrane proteins required for electrical signaling in biology. Naᵥs open their ion-conducting pore and selectively conduct sodium ions in response to membrane depolarization-two features which shape the action potential waveform required for long range signaling in our nervous system¹. There are nine subtypes of the sodium channel in vertebrates (Naᵥ1.1-1.9), which are preferentially expressed in excitable cell types of all organ systems². In mice and humans, Na 1.1 (SCN1A), Naᵥ1.6 (SCN8A), Naᵥ1.7 (SCN9A), Naᵥ1.8 (SCN10A), and Naᵥ1.9 (SCN11A) are expressed by adult sensory neurons³⁻⁶. The dorsal root ganglia (DRG) contain the cell bodies of sensory neurons, and are located adjacent to the spinal column. As such, these primary sensory neurons receive various stimuli from the body and are responsible for the transmission of peripheral sensory information to the central nervous system, making their first synapse in the dorsal horn of the spinal cord. Among the sensory cell types found in DRGs are Ab, Ad and C-fiber afferent neurons, which have unique but sometimes overlapping roles in mechanosensation, thermosensation, and nociception. The unique expression of Naᵥ subtypes found within sensory neurons, along with channel-specific gating properties, define their functional role in pain signaling⁷. Aberrant Naᵥ expression patterns after traumatic injury and inflammation contribute to hyperexcitability of sensory neurons observed in chronic pain states. Furthermore, gain of function gene variants in Naᵥ1.7, Naᵥ1.8, and Naᵥ1.9 are associated with rare pain conditions such as inherited erythromelalgia and paroxysmal extreme pain disorder⁸⁻¹⁰. Only Naᵥ1.7 loss of function gene variants, however, are reported to contribute to pain insensitivity in rare human conditions^{11,12}. These genetic findings establish sensory Naᵥ subtypes as therapeutic targets for pain management.

Chronic nociceptive pain is complex and often poorly understood, yet it affects more than 50 million patients every year¹³. Current standard of care medications to treat moderate to severe nociceptive pain include opioids, adjuvants (e.g. non-steroidal anti-inflammatory drugs, NSAIDs) and psychotropic medications (e.g. tricyclic anti-depressants, TCAs)¹⁴. Opioids are effective analgesics, but their use is marked by increased drug tolerance and high risk of dependency. While commonly used, NSAIDs and TCAs have limited efficacy and are associated with an array of serious cardiovascular and cognitive side effects^{15,16}. Several sodium channel inhibitors, such as carbamazepine and cannabidiol, are also used for chronic neuropathic pain conditions (e.g. painful diabetic neuropathy) but suffer from low efficacy. However, development of peripheral nerve sodium channel antagonists for future pain medications has garnered considerable investment since Naᵥ pharmacology is not associated with addiction.¹⁷

Modern drug discovery relies on high resolution structural determination of therapeutic targets to aid in the design of a high affinity drug. However, eukaryotic Na s are large transmembrane proteins which are difficult to express in sufficient quantities to conduct condition trials for x-ray crystallography. Advances in cryogenic electron microscopy (cryo-EM) have enabled the determination of several eukaryotic Naᵥ structures, which has helped fill this knowledge gap¹⁸⁻²². In addition, several groups have solved the structures of small prokaryotic Naᵥs bound to prototypic and validated drugs using x-ray crystallography and cryo-EM methods²³⁻²⁶. Analgesic drug receptor sites have been identified within the fenestrations of the Naᵥ pore for lamotrigine, lidocaine, cannabidiol and carbamazepine. The tamoxifen receptor has been identified within the prokaryotic NavMs channel, which occupies a binding pocket near the intracellular gate of the channel and is unlike any other drug-binding site for this target²⁷. Prokaryotic sodium channels are related to eukaryotic Naᵥs but structurally assemble as homotetramers^{28,29}. The larger eukaryotic Naᵥs are pseudoheterotetrameric and likely resulted from gene duplication during its evolution from a common prokaryotic Naᵥ ancestor³⁰. There are differences in prokaryotic and eukaryotic Naᵥ functional kinetics and pharmacology, however, such as inactivation rates and tetrodotoxin sensitivity.

Annually, more than 100 million people worldwide are prescribed tamoxifen for the treatment of breast cancer. Tamoxifen primarily targets the estrogen receptor (ER) expressed in adenocarcinoma cells and promotes tumor apoptosis through inhibition of protein kinase C and downstream DNA synthesis pathways^{31,32}. Tamoxifen is presystemically metabolized into N-desmethyl tamoxifen (ND-Tam) and 4-hydroxytamoxifen (4OH-Tam) by cytochrome P450 enzymes in the liver^{33,34}. Importantly, ND-Tam has ˜100 times lower affinity for the ER than 4OH-Tam³⁵. Thus, for this study, the inventors primarily focus on testing ND-Tam pharmacology against sensory neuron Naᵥs to avoid off-target effects associated with ER antagonism.

### Results

Potency Against Endogenous and Heterologously Expressed Naᵥs.

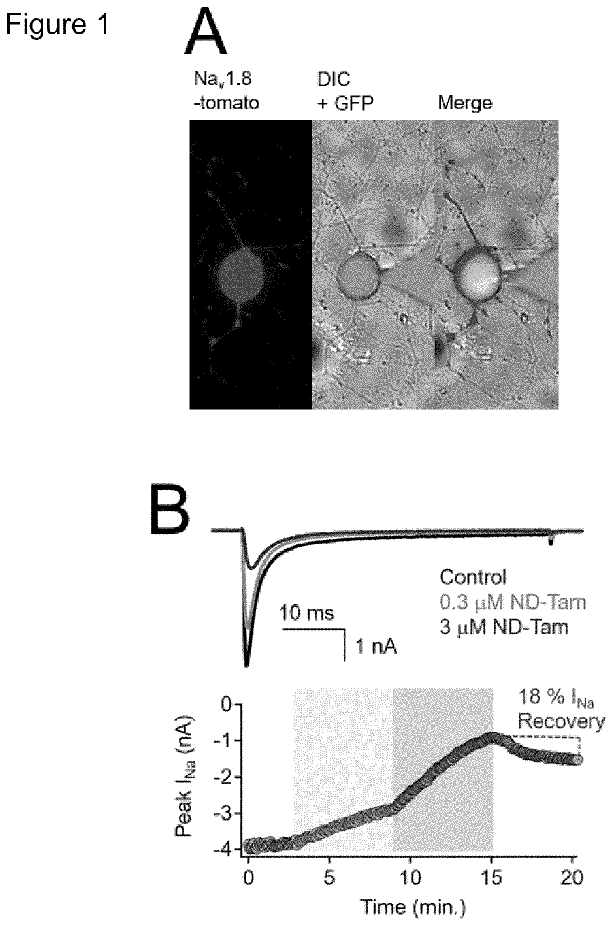

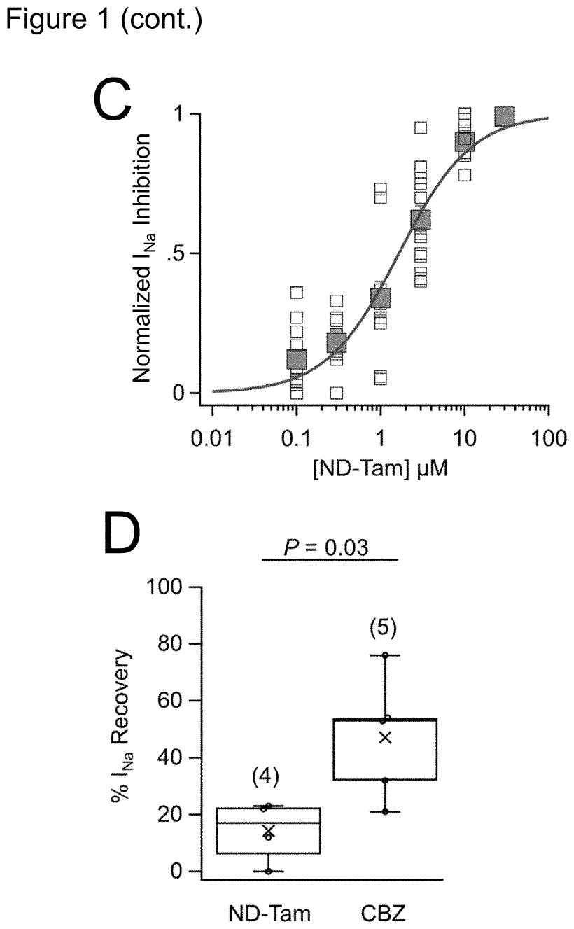

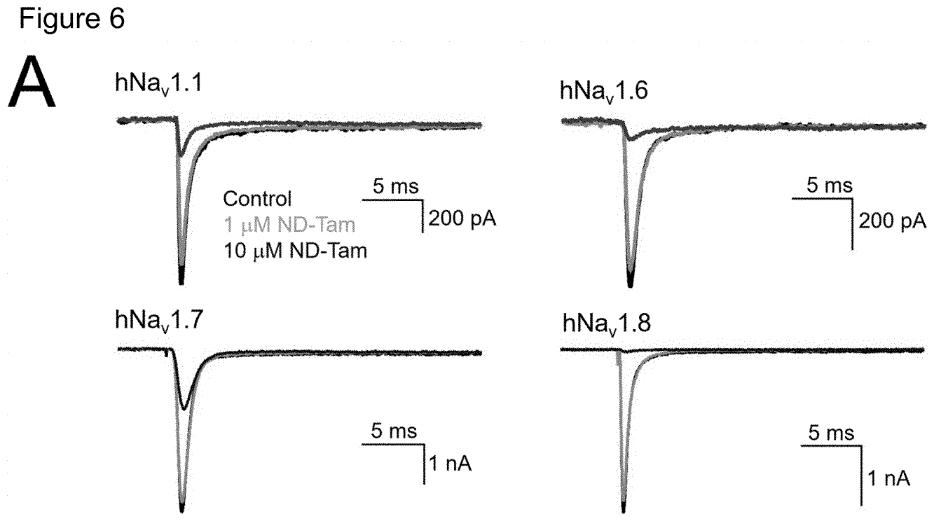

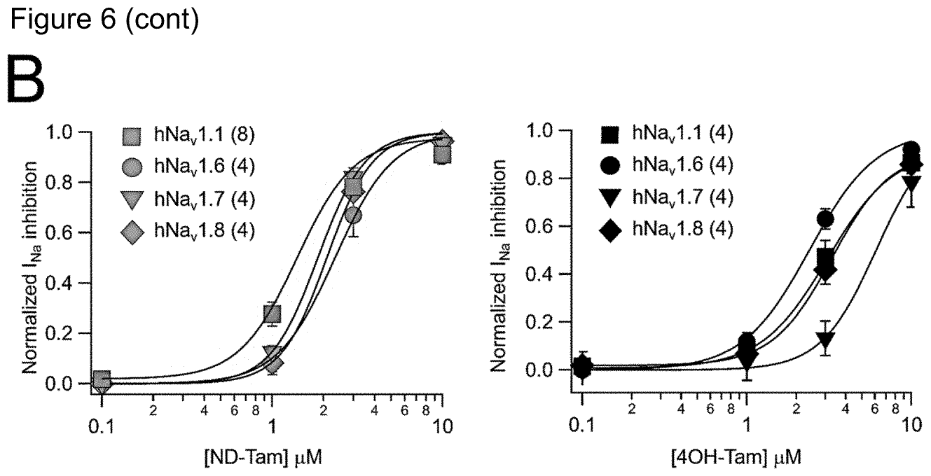

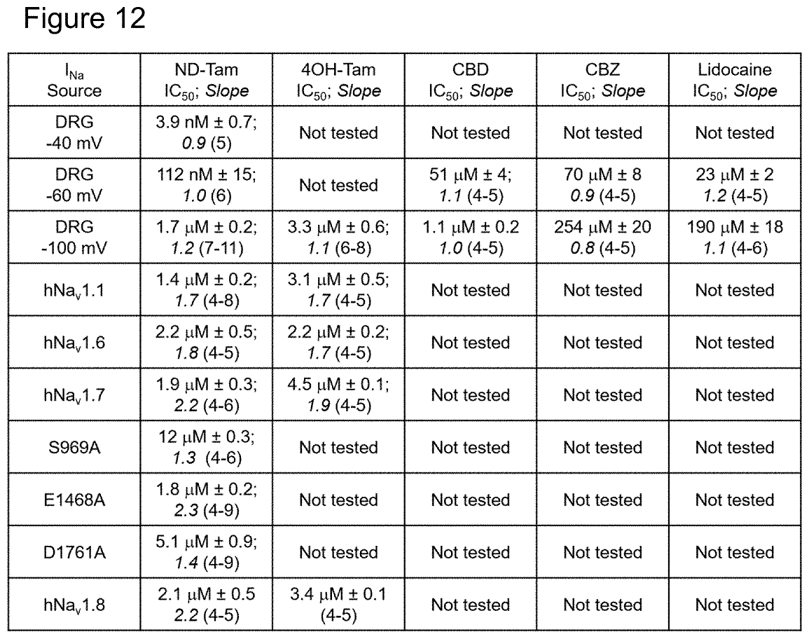

Previously, a unique receptor site for tamoxifen analogues within the prokaryotic sodium channel isolated form Magnetococcus marinus (NavMs)³⁶ (Sula et al) was crystallographically identified. Because drug occupancy of this receptor caused long lasting inhibition of the NavMs I_{Na}, the inventors wondered if these properties would translate into effects against sensory neuron Na s which transmit pain signals. Accordingly, =the potency of ND-Tam and 4OH-Tam was tested against endogenously expressed Na s in cultured DRG sensory neurons isolated from Naᵥ1.8-tdTomato mice by conducting whole cell voltage clamp recordings (FIG. 1A, B). Sodium currents were activated by a 0.2 Hz train of −10 mV depolarizations from a holding potential of −100 mV. The externally applied drug effect was determined after three to five minutes of application of each drug concentration (FIG. 1B). The half maximal potency of I_{Na} inhibition (IC₅₀) for ND-Tam and 4OH-Tam was 1.7 μM±0.2 and 3.3 μM±0.6, respectively (FIG. 1C, FIG. 12). Interestingly, the majority (86±5%) of I_{Na} did not recover after removing ND-Tam from the bath and waiting for 5 minutes (FIG. 1B, D), which suggests that the drug-receptor interaction has a slow dissociation constant. As discussed in the introduction, total DRG I_{Na} is conducted by the Naᵥ1.1, Naᵥ1.6, Naᵥ1.7, Naᵥ1.8 and Naᵥ1.9 subtypes. To determine if there is any specificity in the potency of tamoxifen analogues against Naᵥ subtypes, human orthologues were heterologously expressed in cell lines (HEK and CHO co-expressing hb1 subunits), and conducted voltage-clamp experiments, as previously performed in the DRG neurons. Four of the five channels produced robust voltage-gated sodium currents when expressed separately, but the inventors did not detect currents from cells expressing hNaᵥ1.9, which is possibly due to impaired membrane trafficking with these cells (FIG. 6A)³⁷. Nominal differences in potency of 4OH-Tam and ND-Tam among the human Naᵥ subtypes (FIG. 6B) were observed, where IC₅₀s ranged from 2.2-4.5 mM for 4OH-Tam and 1.4-2.9 mM for ND-Tam when a −100 mV holding potential was applied (FIG. 12). Taken together, these results demonstrate that ND-Tam and 4OH-Tam exhibit potency against the DRG sodium current but with little selectivity for the Naᵥ channel subtypes expressed in sensory neurons.

Conserved Naᵥ Tamoxifen Receptor Sites

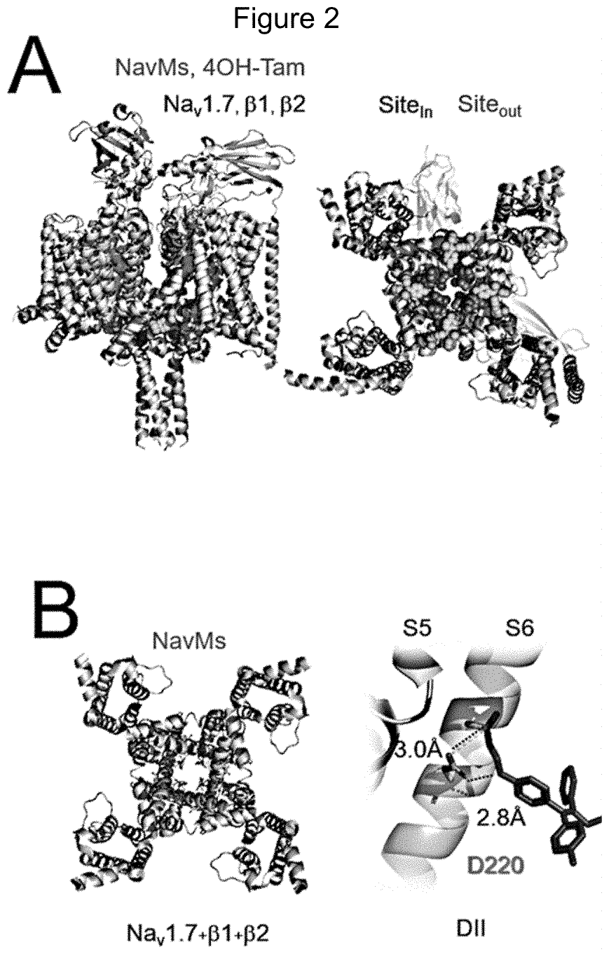

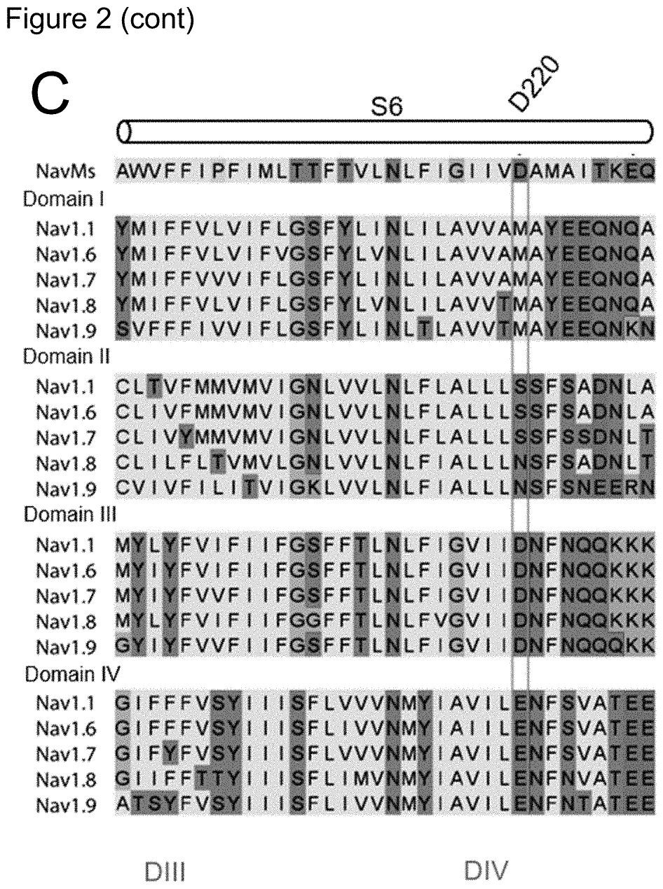

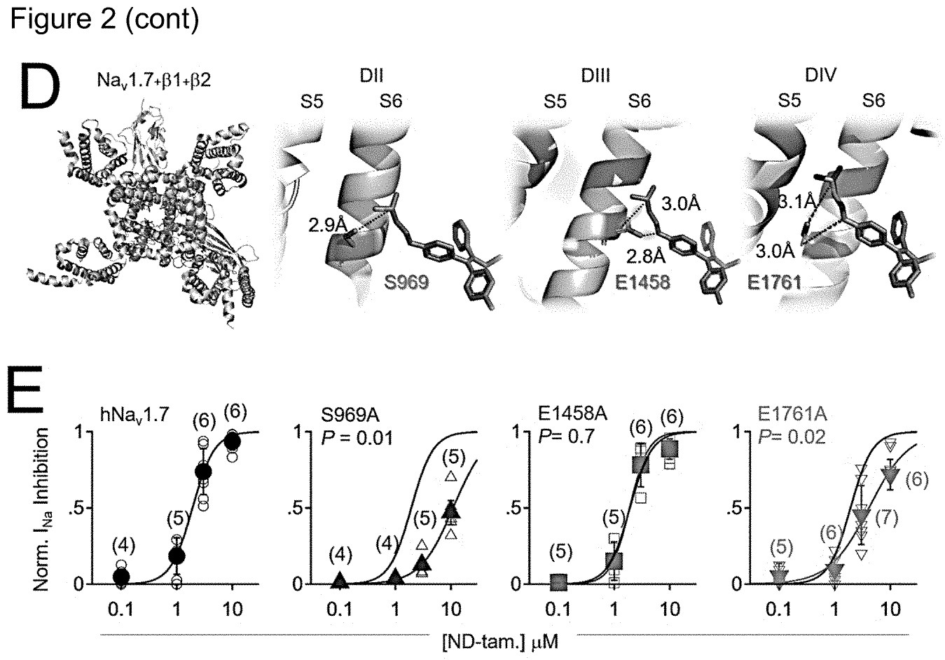

Published crystal structures of the prokaryotic NavMs channel complexed with tamoxifen analogs (Tam, 4OH-tam, endoxifen, ND-Tam) clearly showed the inner (Siteᵢₙ) and outer drug receptor sites (Siteₒᵤₜ) near the channel gate (FIG. 2A) (Sula et al.). While no clear drug-receptor interactions were determined within Siteₒᵤₜ, the S6 E220 side chains formed hydrogen bond interactions with the ether and amine moieties of the ND-Tam molecules found at Siteᵢₙ. Alignment of NavMs with human sensory neuron voltage gated sodium channels (Naᵥ1.1, Naᵥ1.6, Naᵥ1.7, Naᵥ1.8, Naᵥ1.9) suggest that drug interactions with the S6 of NavMs are conserved in domain two (DII), three (DIII) and four (DIV) (FIG. 2B). After structural alignment of the crystalized NavMs-Tam complex with the cryo EM Naᵥ1.7+b1+b2 channel coordinates, the S6 side chain hydroxyl in DII (S969) and the carboxyl side chains in DIII (E1458) and DIV (D1761) observed in the human channel were nearly superimposable with E220 of the prokaryotic Naᵥ subunits. In addition, the distances from the drug ether and amines were within acceptable distances (2.8-3.1 Å) for hydrogen bond formation for all three site chains (FIG. 2B). ND-Tam and 4OH-Tam inhibited I_{Na} from heterologouly expressed Naᵥ1.7 (IC₅₀=1.9-4.5 mM) with a steep concentration-dependence (slope=2.1), which suggests two drug binding sites within the channel. To determine which of the three domain receptor sites coordinate ND-tam molecules and inhibit Naᵥ1.7 I_{Na}, the inventors independently neutralized each of the residues with alanine substitutions. For the DII S969A and DIV D1761A sites, reduced potency of I_{Na} inhibition (IC₅₀=5.1-12 mM) and a reduction in the slope (1.3-1.4) was observed, whereas no change in potency (IC₅₀=1.3 mM) or slope (2.2) was observed for D1458A in domain III (FIG. 2D, FIG. 12). These data demonstrate that the S6 NavMs-tamoxifen receptor site is conserved in DII and DIII of Naᵥ1.7, which is located near the exit of the Na⁺ conducting pathway into the cell. The mechanism by which ND-Tam occupancy results in Naᵥ inhibition is considered in the next section.

Structure-Activity Relationship Indicates ‘Bind and Plug’ Model

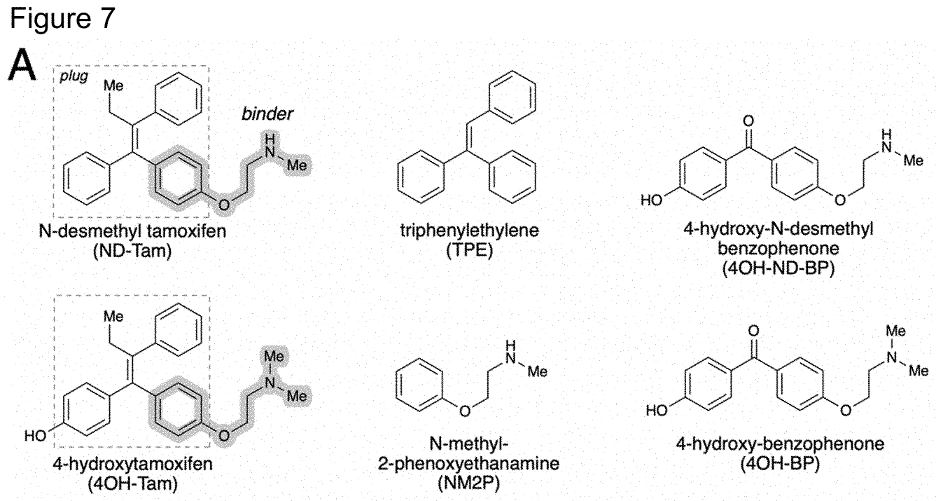

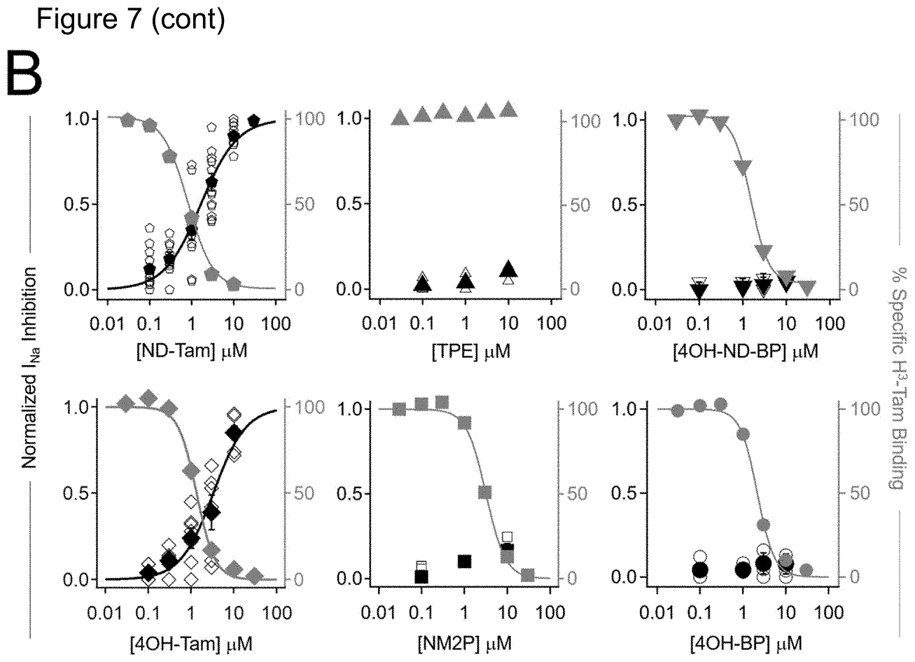

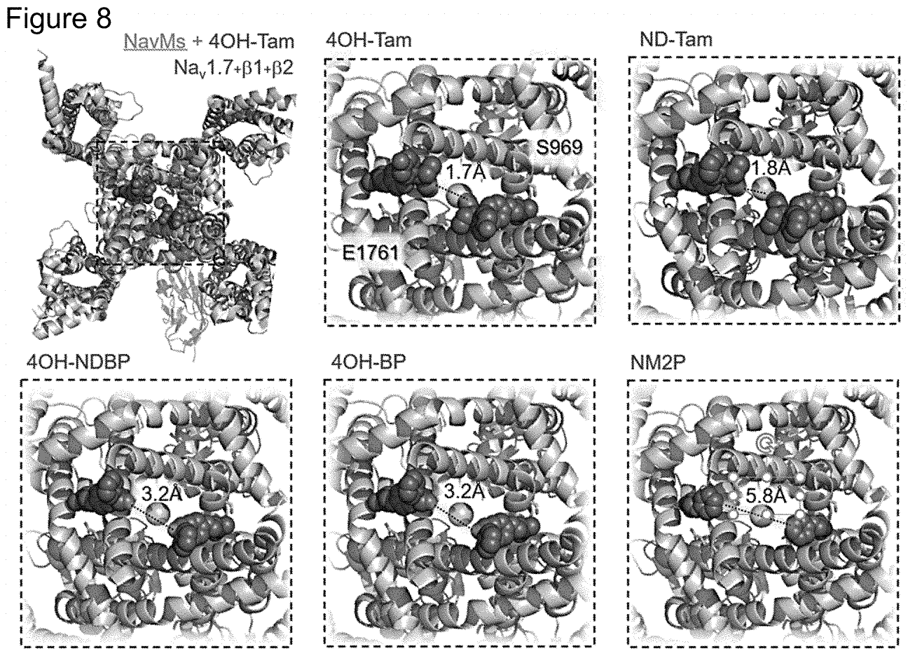

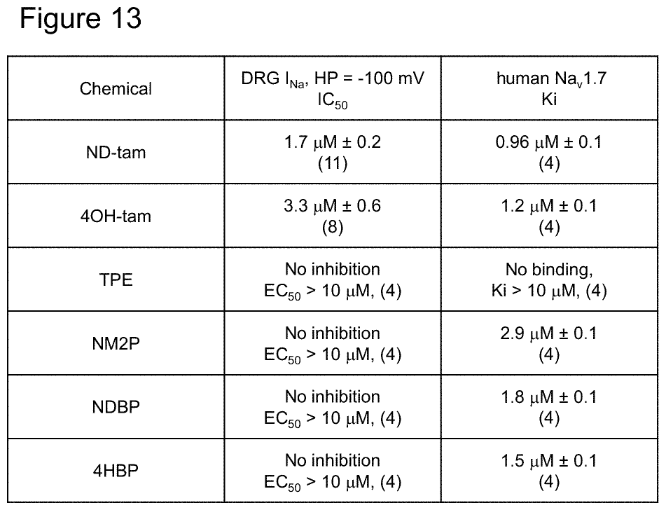

To better understand how tamoxifen analogues inhibit human sodium channels, the inventors tested the direct binding of chemical ND-tam analogs to the Naᵥ1.7 channel, and compared their functional inhibition of sodium currents recorded from DRG neurons (FIG. 7). The parental molecular structure of ND-tam can be separated into the receptor-binding N-methyl 2 phenoxyethanamine (NM2P) and triphenylene (TPE) fragments, called the ‘binder’ and ‘plug’, respectively. These fragments, along with 4OH-tam and synthesized benzophenone analogs lacking the TPE fragment, 4-hydroxy-N-desmethyl benzophenone (4OH-ND-BP) and 4-hydroxy benzophenone (4OH-BP), were tested for affinity for the human Naᵥ1.7 tamoxifen receptor in a competitive binding assay using a tritium labeled tamoxifen (H³-Tam). All analogs have affinity (Ki=0.96-2.9 mM, FIG. 13) with the exception of TPE, which was expected given that the hydrophobic payload does not have defined chemical interactions within the channel structure (FIG. 7A). However, none of these compounds, except for 4OH-tam and ND-Tam, inhibited sodium currents recorded from DRG neurons (FIG. 7B). This was an unexpected result given the demonstrated specific binding to hNaᵥ1.7, presumably because the hydrogen bonds formed between the channel and NM2P, 4OH-ND-BP and 4OH-BP, should be preserved. These data suggest that Naᵥ occupancy at the receptor site alone does not produce sodium current antagonism. By substituting the tamoxifen molecules found in DII and DIV of the NavMs-hNaᵥ1.7 aligned structures with the inert drug fragments, the inventors observed that the TPE hydrophobic plug moiety of 4OH-Tam and ND-Tam molecules encroach into the 2 Å diameter of the ion-conducting pathway. Whereas analogs NM2P, 4OH-ND-BP and 4OH-BP may occupy DII and DIV receptor sites but leave the pore unobstructed (FIG. 8). Based on this limited SAR data set, the inventors propose that the efficacy of the analogues is dependent on the combination of the ‘channel-binding’ NM12P chemical moiety with a pore-blocking TPE ‘hydrophobic plug’. Features of tamoxifen pharmacology against neuronal sodium channels which enhance its potency and specificity are considered in the next section.

State-Dependent Naᵥ Inhibition in DRGs.

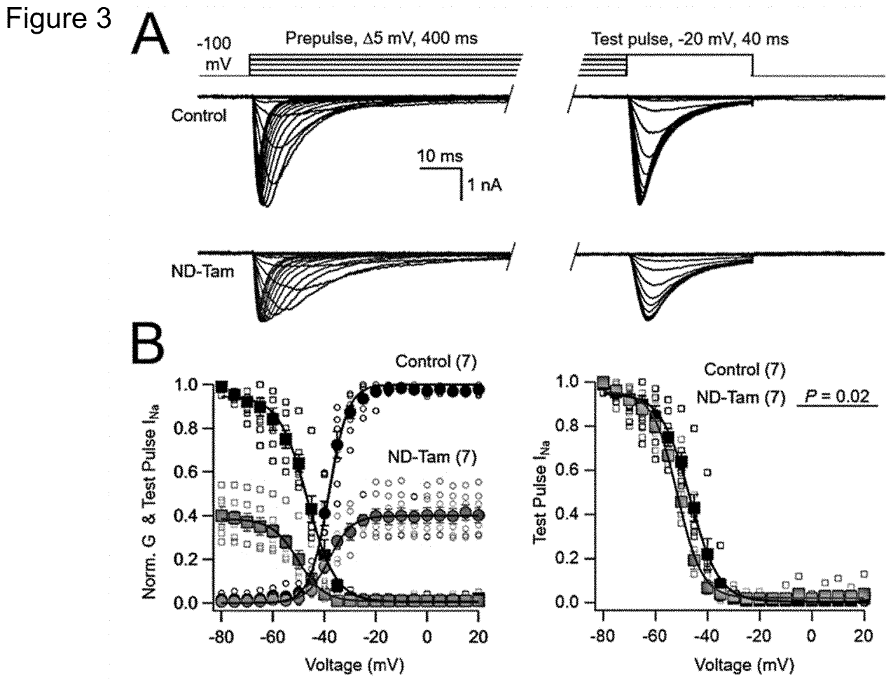

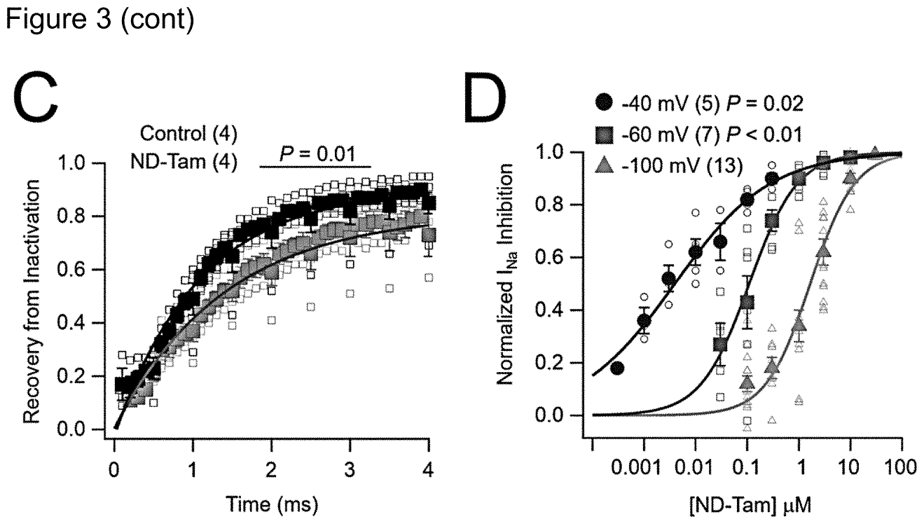

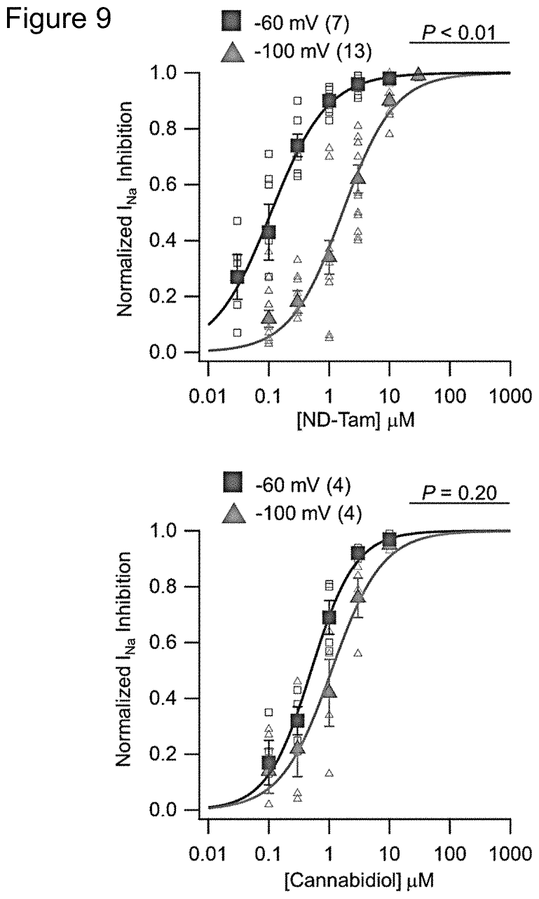

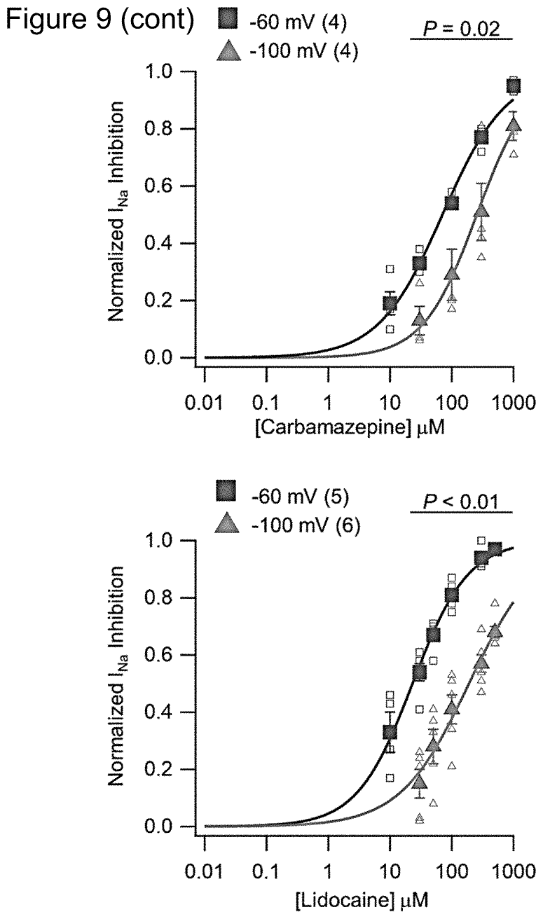

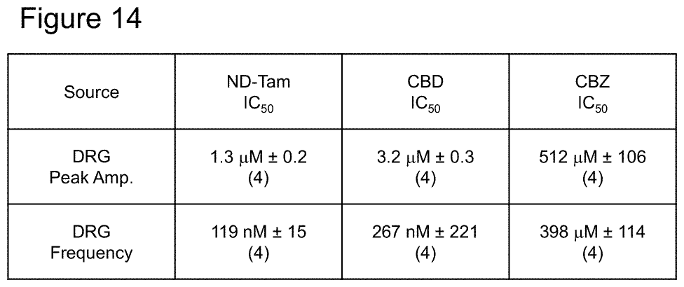

Naᵥs undergo changes in structural conformation when neuronal membranes are depolarized. Here, Naᵥs transition from the closed to open and inactivated states, which ultimately initiates and terminates the repetitive depolarizing peak waveforms (spiking behavior) observed in sensory neuron action potentials. State-dependent accessibility of receptor sites by analgesic drugs provides a selective block of Naᵥs expressed in hyperexcitable sensory neuronal circuits—a mechanism which is postulated to contribute to their clinical efficacy^{38,39}. As previously reported, the primary mechanism of NavMs inhibition by tamoxifen, and its analogues, is stabilization of the non-conducting inactivated channel state, where the recovery rate from this non-conducted state is delayed by ≈10×. However, when tested against DRG neurons, 3 μM ND-Tam had nominal impact on the recovery time from inactivation (τ_{rec.}=1.1±0.1 ms) when compared to untreated neurons (τ_{rec.}=1.4±0.1 ms) (FIG. 3C). The inventors also examined the effects of ND-Tam on Naᵥ steady state voltage-dependent conductance (GV_{1/2}) and inactivation (Inact. V_{1/2}) after blocking ≈45% of the total I_{Na} with 3 μM ND-Tam treatment. No change in the half-maximal activation of conductance (GV_{1/2}) was observed, which suggests that ND-Tam prevents DRG Naᵥs from conducting without altering activation (FIG. 3A, B). However, a significant (P=0.01) shift in voltage dependence of inactivation by −8 mV after ND-Tam treatment was observed, suggesting that drug affinity may be enhanced in the Naᵥ inactivated state. To test the proposed mechanism of action, ND-Tam potency against DRG I_{Na} was assessed over more depolarizing holding potentials (−40, −60 and −100 mV) and the inventors observed a 15-44× increase (IC₅₀=3.9 nM±0.7, 112 nM±15 and 1.7 μM±0.2, respectively) when neuron membranes were held at more depolarizing potentials (FIG. 3D, FIG. 12). For comparison of ND-Tam effects among clinically used compounds reported to have voltage-dependent shifts in potency against Naᵥs, we also tested carbamazepine (CBZ), lidocaine, and cannabidiol (CBD)⁴⁰⁴¹. Inhibition by CBZ and lidocaine was most potent when membranes were held at −60 mV (FIG. 9, FIG. 14). CBD followed this trend but the sample size lacked the statistical power to test significance. None of these compounds were as effective I_{Na} antagonists as ND-Tam, however, which reached half maximal inhibition at 112 nM using a −60 mV holding potential (FIG. 12). Taken together, these findings indicate that tamoxifen's mechanism of action against prokaryotic and human sensory neuron Naᵥs are divergent. The implication of ND-tam inhibition of sensory neuronal transmission and in vivo efficacy are considered in the following sections.

Inhibition of DRG Neuronal Transmission.

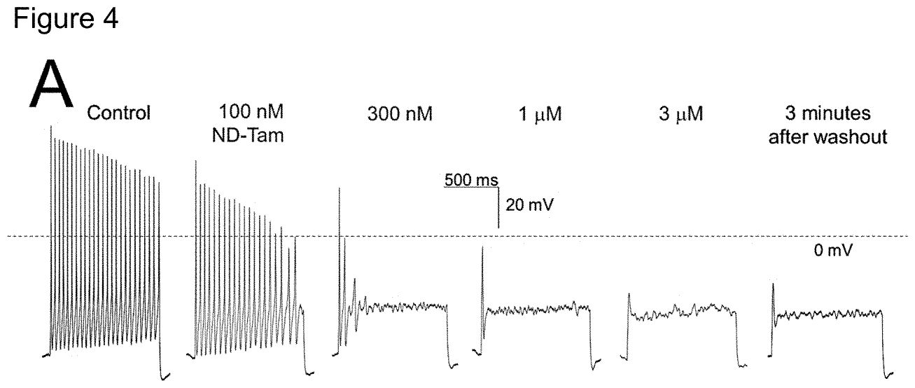

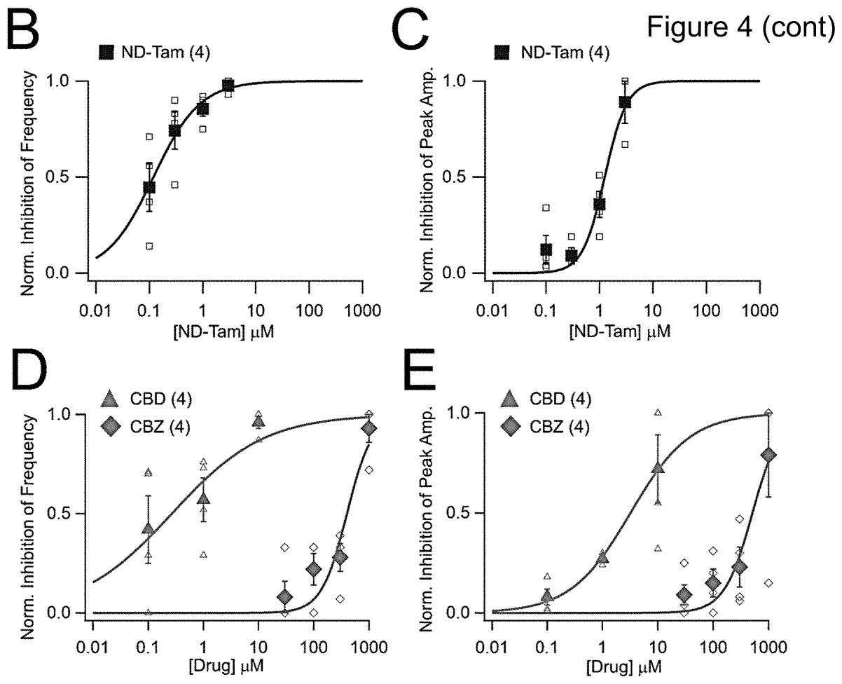

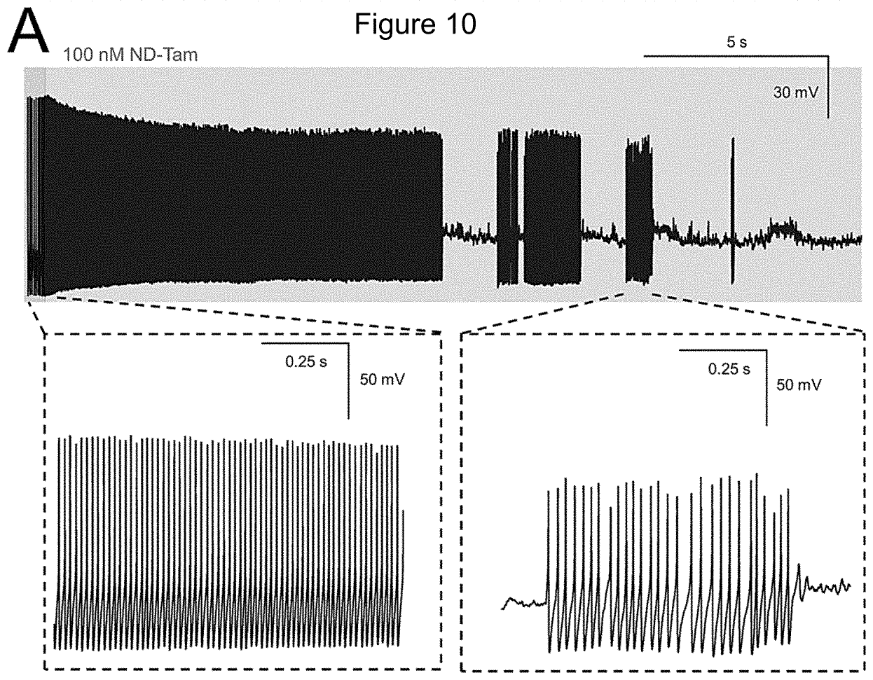

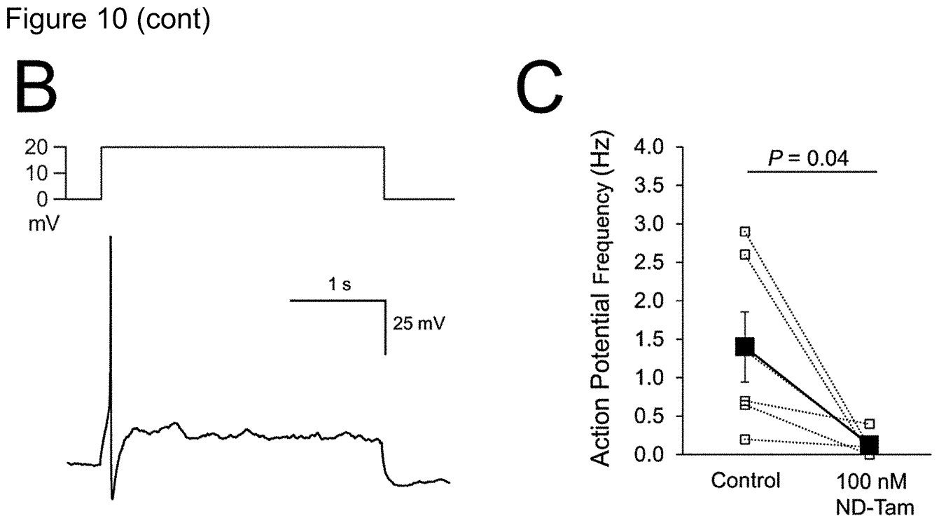

DRG neurons act as a filter to electrical information from the periphery, required for the perception of pain, generated by several types of ion channels, including Naᵥs. For that reason, the inventors compared the pharmacology of ND-Tam against cultured DRG action potentials recorded using current clamp (FIG. 4A). The inventors then compared DRG action potential frequency and amplitude before and after 2-4 minutes of extracellular drug treatment (FIG. 4A-C, FIG. 14). ND-Tam was most potent against the firing frequency (IC₅₀=119 nM±15) elicited by 80 pA of injected current, which recapitulated potency observed against DRG I_{Na} recorded with a −60 mV holding potential (FIG. 3D, FIG. 9). ND-Tam also inhibited the peak amplitude (IC₅₀=1.3 μM±0.2) of the rapid depolarizing phase of the action potential carried by I_{Na} (FIG. 4C). After exchanging the drug for control saline for 2 minutes, ND-Tam inhibition of action potential frequency and peak amplitude was persistent (FIG. 4A), which is consistent with the observed slow dissociation of the drug with sodium channels (FIG. 1B, D). Additionally, as observed against sodium currents, ND-Tam was a more potent inhibitor of DRG action potential frequency and peak amplitude than CBZ and CBD (FIG. 4D, E, FIG. 14). Approximately 10% (11/112) of the cultured neurons exhibited spontaneous action potentials. The inventors excluded this data from the aforementioned analyses and tested a low dose (100 nM) of ND-Tam against these aberrant action potentials (FIG. 10A, B). Interestingly, a three-minute extracellular application of 100 nM ND-Tam reduced the frequency of the spontaneous sensory neuron action potentials by 91% (n=6, P=0.04) (FIG. 10C). This result suggests that at low doses, ND-Tam is an effective inhibitor of aberrant firing of hyperexcitable neurons—a feature likely conferred by its high degree of voltage-dependent inhibition of sodium channels (FIG. 3D). Taken together, these results clearly demonstrate that ND-Tam is an effective inhibitor of neurotransmission and that Naᵥ subtypes expressed in these sensory neurons are the likely primary molecular target.

Efficacy of ND-Tam Ameliorating Arthritic Pain In Vivo.

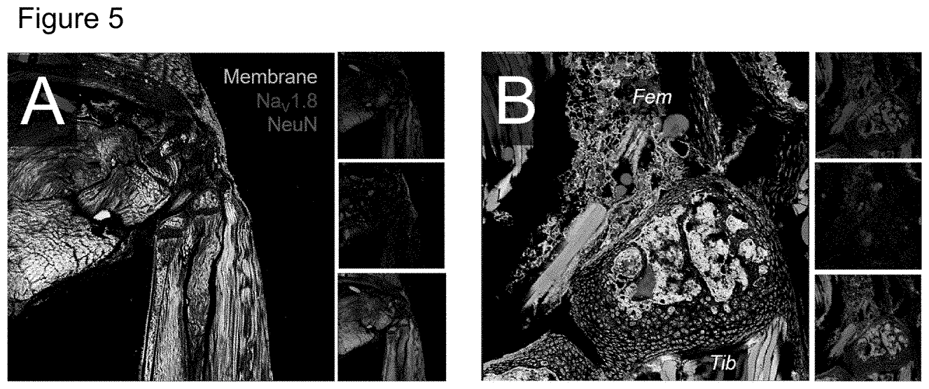

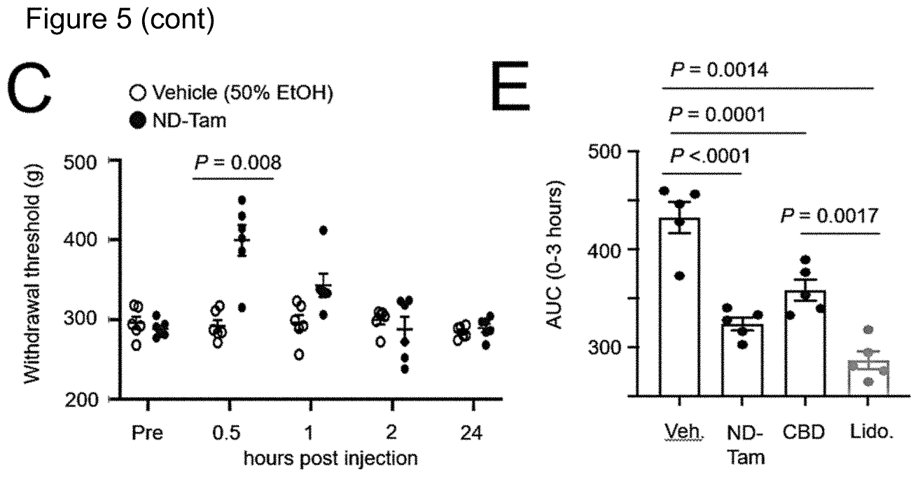

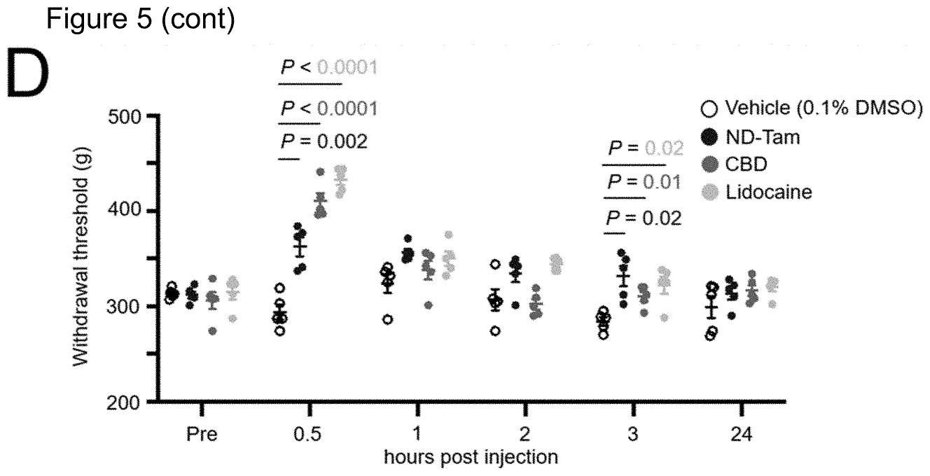

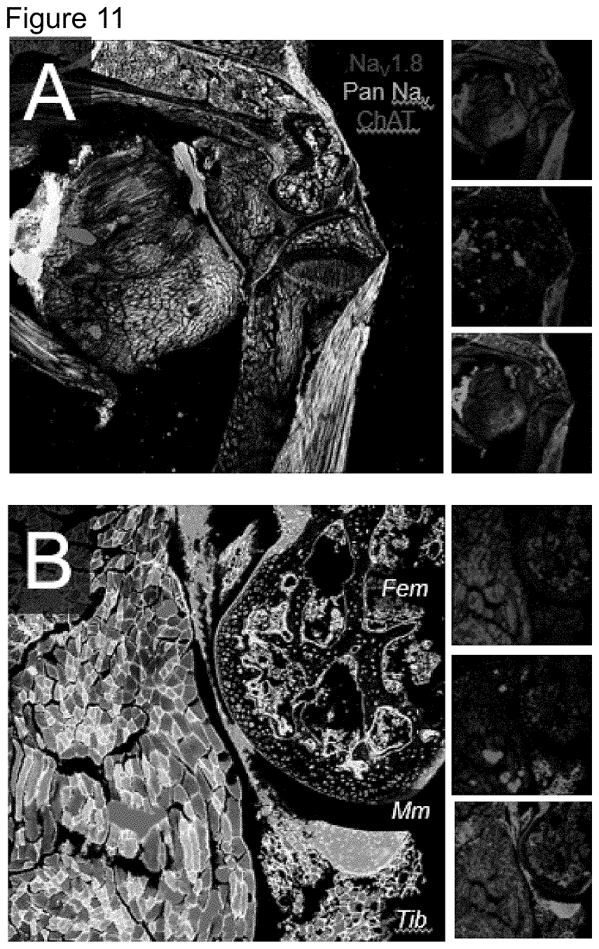

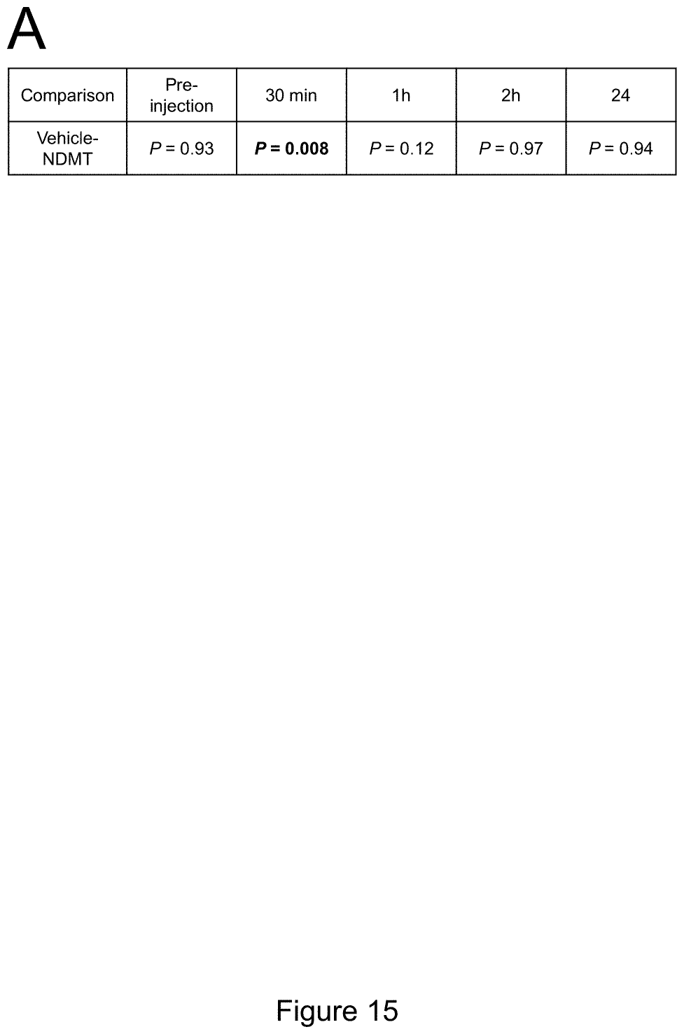

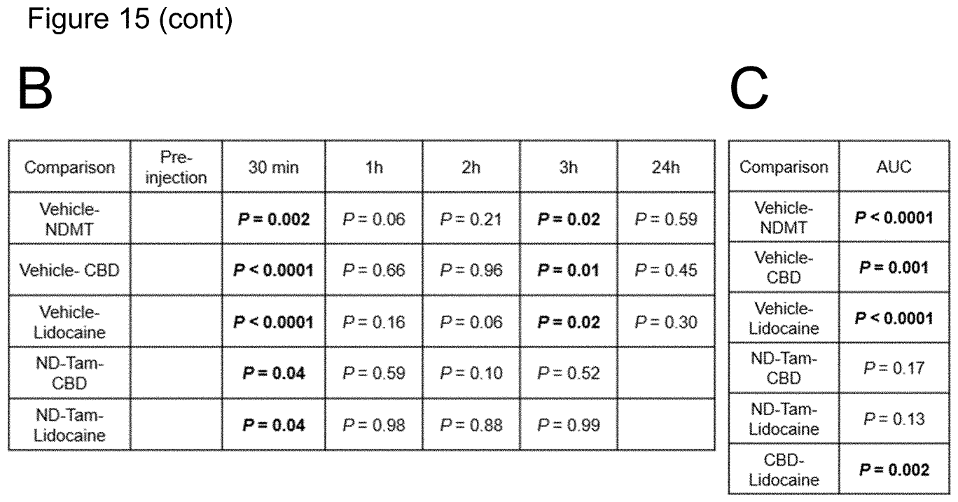

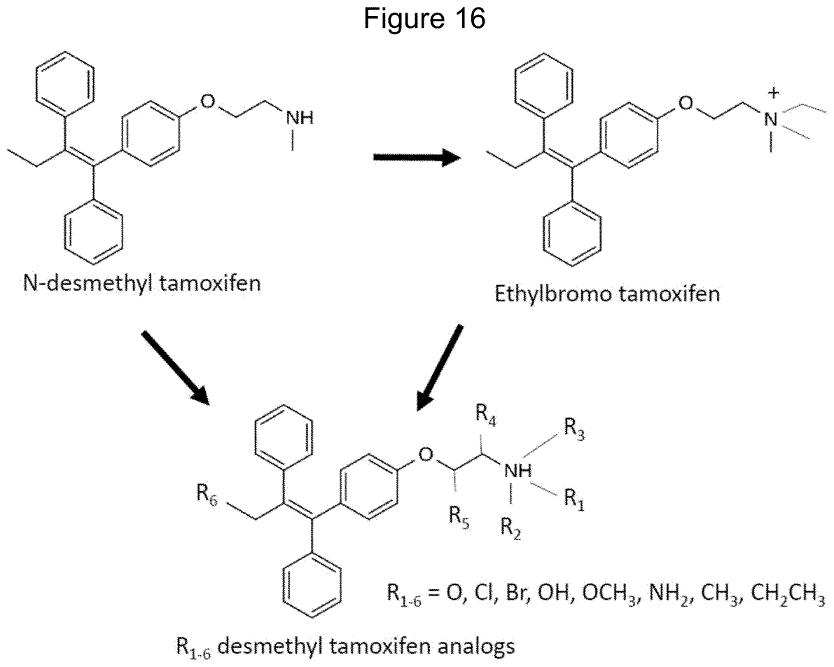

Given the high potency Naᵥ inhibition demonstrated in cultured DRG sensory neurons, the inventors wanted to investigate whether ND-Tam had analgesic effects against nociceptive pain in vivo. To do this, an osteoarthritis mouse model was used where destabilization of the medial meniscus (DMM) induces osteoarthritis and associated pain. As previously shown, Naᵥ1.8-tomato positive sensory neurons are present in the synovial joint as visualized by immunohistochemistry (FIG. 5A, B, and FIG. 11) (PMID: 33827672, PMID: 31351964). Previously, the inventors demonstrated that remodeling of Naᵥ1.8+ nerves in the knee joint is a key part of the pathogenesis process of osteoarthritis (PMID: 31351964) and local injection of a relatively high dose of lidocaine (20 mg/kg, ˜0.85M) was able to acutely reverse knee hyperalgesia (PMID: 29563338). Here, the inventors were interested to test whether ND-Tam could also inhibit knee pain, but at a lower dose. Local injection of 50 μM ND-Tam (˜0.002 mg/kg) into the knee joint rapidly inhibited knee hyperalgesia pain compared to injection of vehicle (P=0.008, 30 minutes after injection) (FIG. 5C, FIG. 15). Therefore, the inventors were interested to compare the efficacy of this low dose of ND-Tam to similar low doses of CBD and lidocaine. In a direct comparison study, local injection of 50 μM ND-Tam (˜0.002 mg/kg), CBD (˜0.002 mg/kg), or lidocaine (˜0.001 mg/kg) were found to all effectively inhibited knee hyperalgesia 30 minutes after injection (p=0.002, p<0.001, and p<0.001 vs. vehicle, respectively) and remained elevated compared to vehicle control through 3 hours after injection (p=0.02, p=0.01, and p=0.02 vs. vehicle, respectively) (FIG. 5D, FIG. 15B). Area under the curve analysis of the first 3 hours post injection also demonstrated similar efficacy of all 3 drugs (ND-Tam, CBD, lidocaine) compared to vehicle (p<0.0001, p=0.0012, p<0.0001 vs. vehicle, respectively) (FIG. 5E, FIG. 15C). These results support the potential therapeutic development of ND-Tam for pain relief.

### Discussion

The inventors have determined the pharmacology of tamoxifen analogs against sensory neuron Naᵥs, both in terms of their molecular receptor interactions and drug chemical properties required for their efficacy. The inventors have focused on the properties of ND-Tam, a first pass metabolic product of tamoxifen that has nominal efficacy against the estrogen receptor. Through binding, structural alignments and functional data sets, the inventors establish that ND-Tam antagonizes sodium current by directly blocking the intracellular pore of Naᵥ1.7 at a previously uncharacterized receptor site within domains II and IV. In DRG sensory nerves, ND-Tam is a more potent Naᵥ antagonist of sodium currents and neuronal transmission than carbamazepine and cannabidiol, two clinically used antagonists to treat chronic nociceptive pain. ND-Tam preferentially inhibits sodium channels in the open/inactivated state, a feature observed in clinically useful analgesics and anti-epilepsy medications. This property confers voltage- and frequency-dependent drug inhibition Naᵥs which preferentially blocks I_{Na} from damaged and/or hyperexcitable neurons, as supported by our DRG current clamp results. The inventors demonstrate ND-Tam is efficacy in ameliorating pain in a rodent osteoarthritis arthritis model when injected into the synovial joint, demonstrating its potential for further development and the possibility of repurposing tamoxifen analogues for the treatment of nociceptive pain. Results from the described SAR study suggest that efficacy against Naᵥs by Tam analogues is tied to the union of two chemical properties: the affinity of the receptor-binding moiety and the hydrophobic bulk of the pore-blocking ‘plug’.

Despite the clear need, the time and cost of developing new analgesics is escalating^{42,43}. Currently, bringing a new drug to market requires an average of ten years to develop, and typically costs more than three billion dollars. High attrition rates among new drug candidates and changing regulatory requirements contributes to this trend⁴⁴. Thus, there are clear benefits to repurposing tamoxifen analogues for the treatment of nociceptive pain. Tamoxifen drug safety, metabolism and distribution is well characterized by several decades of use as treatment for breast cancer. Thus, the short- and long-term potential drug toxicity, pharmacokinetics and pharmacodynamics associated with orally-dosed tamoxifen is already established within a large, heterogenic human population.

There are currently effective therapeutics for acute nociceptive pain, but the greatest unmet medical need is chronic pain associated with specific diseases. Musculoskeletal pain in particular has repeatedly been highlighted as an unmet medical need and is a leading cause of global burden of disease (PMID: 30496104). For diseases such as osteoarthritis with peripheral sources of pain, in this case ongoing tissue destruction in the knee joint, targeting Naᵥs can be therapeutically useful since it is the common mediator of chronic and acute pain transmission.

### Methods

Isolation and primary culture of marine dorsal root ganglia sensory neurons.

Dorsal root ganglia were isolated using a modified version of the previously described protocol (PMID: 26864470). All animal procedures were approved by the Institutional Animal Care and Use Committee at the Feinberg Medical School, Northwestern University. The spinal columns of 4-6-week-old C₅₆/B6 or Naᵥ1.8-Tomato mice (Naᵥ1.8-Td) were removed and DRG neurons from all spinal levels were isolated for primary culture (PMID: 33827672, PMID: 31351964). The collected DRGs, containing both neuronal and non-neuronal cells, were acutely dissociated in DMEM with collagenase IV (2 mg/mL) for 30 minutes, followed by papain (25 U/mL) for 30 minutes. The cells were then triturated, filtered through a 40 μm cell strainer to remove non-dissociated cells, and washed with DMEM. The DRGs were resuspended in 70 μL of pre-warmed growth medium (DMEM F12+Glutamax supplemented with 0.5% FBS, 0.5% penicillin/streptomycin, 1% N-2 supplement, and β-NGF (5 ng/μL) added on the day of use) and plated on poly-l-lysine (10 mg/mL) and laminin (25 μg/mL) prepared coverslips. The DRGs were incubated at 37° C. for 2-4 hours before more growth medium was added to the wells. The cells were then allowed to adhere to the coverslip undisturbed at 37° C. for 12-48 hours, until used for electrophysiology recordings.

Electrophysiology of Endogenous and Heterologously Expressed Sodium Channels.

Plasmids encoding the human version of channels Naᵥ1.1, 1.6, and 1.7 were co-transfected with TRES GFP into HEK cell lines for whole-cell voltage clamp studies (American Type Culture Collection). Transient transfections were performed using lipofectamine 24-48 hours prior to electrophysiology recordings. Chinese hamster ovary (CHO) cells stably expressing human b1 and b2, were transiently transfected with plasmids encoding for either human Naᵥ1.8 or Naᵥ1.9 plasmids alpha subunits were used to record sodium currents from these channel subtypes. Plasma membrane currents were recorded using borosilicate glass electrodes polished to resistances of 2-4 mW. Sodium currents conducted by heterologously expressed Naᵥs were recorded using the following solutions (in mM). The internal (pipette) solution contained 110 CsF, 30 NaCl, 10 HEPES, and 5 mM EGTA (ethylene glycol-aminoethyl ether-N,N,N0,N0-tetraacetic acid) and the pH was adjusted to 7.3 using CsOH. The extracellular (bath) solution contained 150 NaCl, 10 HEPES, 1.8 CaCl₂, and pH was adjusted to 7.4 using NaOH. The osmolality of these solutions were adjusted to 300 mOsm using mannitol. Endogenous DRG sodium currents were recorded using an intracellular solution consisting of 70 CsCl, 70 CsF, 2 EGTA, 5 HEPES, and 5 NaCl; the pH was adjusted to 7.4 with CsOH. The extracellular solution contained 125 NaCl, 25 glucose, 20 TEA-Cl, 1 MgCl₂, 1.8 CaCl₂, 5 HEPES, and 5 CsCl; the pH was adjusted to 7.4 with TEA-OH. DRG action potentials were measured in current clamp mode using an intracellular solution containing 140 KCl, 10 HEPES, 5 MgCl₂, 5 EGTA, 2.5 CaCl₂, 4 MgATP, 0.3 GTP, and the pH adjusted to 7.3 using KOH. The extracellular solution contained 140 NaCl, 10 HEPES, 10 glucose, 5.3 KCl, 1 MgCl₂, 1.8 mM CaCl₂ and the pH adjusted to 7.3 using NaOH. For both voltage and current clamp recordings in DRGs, the intracellular solution osmolality was 280 mOsm and the extracellular solution was adjusted to 325 mOsm with mannitol. Voltage and current clamp data were collected using Multiclamp and Axopatch 200B amplifiers supplied by Molecular Devices. Analog signals were converted to digital signals using a Digidata 1550B and controlled using pClamp 10 software. Currents were digitized at 25 kHz and low-pass filtered at 5 kHz. All drug stocks were formulated in DMSO at 10 or 100 mM and stored at −20 C.° until the day of use. All drug stocks were then diluted into extracellular saline solutions. Drugs were applied using a gravity fed extracellular bath perfusion system with a flow rate of 5-10 ml/minute through a 0.4 ml volume recording chamber.

Current clamp and voltage clamp data were analyzed using Clampfit (Molecular Devices) and IGOR Pro 8.1 (Wavemetrics). Leak current was subtracted using a standard P/-4 protocol. Data from cells whose leak current exceeded −150 pA at −100 mV or whose voltage error exceeded 10 mV were excluded from the final analysis. Series resistance (Rs) was compensated by at least 80% to limit Rs related error to <3 mV. Rs was monitored periodically throughout the experiment to check for shifts in voltage error. Normalized I_{Na} inhibition was determined by taking the ratio of the current at steady state drug block (I_{drug}) and control current (I_{control}), and expressed as: Normalized I_{Na} inhibition=1−(I_{drug}/I_{control}). Normalized I_{Na} inhibition was plotted as a function of drug concentration, and the data were fit with the Hill equation: y=base+(max−base)/[1+(IC₅₀/x)\hat{ }rate], where base and max describe the lower and higher asymptotes, respectively; x is the drug concentration and IC₅₀ is the drug concentration that produces a 50% maximal inhibitory response, and rate is the Hill coefficient. Percent current recovery was calculated by (I_{recovery}−I_{drug})×100, where I_{recovery} is the recovered current measured 3-5 min after drug removal. Normalized pre-pulse I_{Na} from FIG. 3 was converted to normalized conductance (G) using the following equation: G=I_{Na}/(Vm−Vᵣₑᵥ), where Vm and Vᵣₑᵥ is equal to voltage applied across the membrane and reversal potential, respectively. The steady state voltage dependence of activation and inactivation was fit to Boltzmann equation: G/Gₘₐₓ or I/Iₘₐₓ=1/(1+exp((V_{1/2}−V)/k)), where V_{1/2} is equal to the half maximal activation (Act. V_{1/2}) or inactivation (Inact. V_{1/2}), and K is equal to the slope. Recovery from inactivation was estimated by fitting the Iₚᵣₑ₋ₚᵤₗₛₑ/Iₜₑₛₜₚᵤₗₛₑ to the exponential equation: f(x)=B+A·exp[(1/τ_{inact.})x], where x is the time between the pre- and test pulse and τ_{inact.} is the half time of total current recovery from inactivation. Gibbs law of free energy was used to calculate the free energy of drug binding: ΔG=−R·T·Ln(Kd), where R=0.008314 kJ/mol, temperature T=297 K, and Kd is the apparent association constant estimated by the IC₅₀.

Naᵥ1.7 Tritium-Labeled Tamoxifen Competitive Binding Assay

Membrane preparations from MDA-MB-231 cells, which are the triple negative (estrogen receptor negative) and infected with lenity virus encoding for human Naᵥ1.7 [Gradek et al., 2019; Brackenbury et al., 2007], were prepared as follows. Cells were grown to 60-80% confluent and harvested with PBS-based, enzyme-free cell dissociation buffer containing EGTA. Cells were then centrifuged at (14000×g, 4° C.) for 15 min. and homogenized (Tekmar Tissuemizer). The cell homogenate was centrifuged (2000×g, 4° C.) for 10 min. Membrane pellets were suspended in 10 ml of PBS/gram and stored at −80° C. Total protein concentration was using the Coomassie (Bradford) method. H³-Tamoxifen was synthesized by Tritech AG (Switzerland). On the day of the experiment, suspended membranes expressing Naᵥ1.7 (final protein concentration=30 mg/well) were added to each of a 96-well plate with 3H-Tam at 30 mM. The plates were incubated at 37 C for 1 hour, aspirated onto filter plates, and rinsed with wash buffer. After addition scintillant (Packard Microscint-20), radioactivity was quantified (Packard Topcount Scintillation Counter). Counts per minute data from binding experiments were converted to percent total specific bound (% TSB) using the following formula: % TSB=[(cpm NSB)/(TB NSB)]100, where TB NSB is the total non-specific binding. Ki values were derived by means of the Cheng and Prusoff (1973) PMID: 4202581 equation (Ki=IC₅₀/1+[analog]/Kd) using Kd values for [3H-Tam] obtained from saturation assays.

Synthesis and Procurement of Naᵥ Antagonists.

N-desmethyl tamoxifen, 4-hydroxytamoxifen, cannabidiol, carbamazepine, N-methyl 2 phenoxyethanamine and triphenylene and were purchased from Sigma Aldrich. The benzophenone 4-hydroxy-tamoxifen analogs, 4-hydroxy-N-desmethyl benzophenone (4OH-ND-BP) and 4-hydroxy benzophenone (4OH-BP) were prepared according to literature procedures and all characterization data matched the literature values.^{46,47}

In Vivo Osteoarthritis Pain Model.

We used a total of n=32 male C57BL/6 mice. All animal procedures were approved by the Institutional Animal Care and Use Committee at Rush University Medical Center. Animals were housed with food and water ad libitum and kept on 12-h light cycles. Surgical destabilization of the medial meniscus (DMM) was performed in the right knee of 10-week-old male mice, as previously described (PMID: 17470400, PMID: 23185004). In experiment 1, a volume of 3 mL of 50 mM ND-Tam formulated in 50% EtOH or vehicle (50% EtOH) was intra-articularly injected into the right knee of mice 4-5 weeks after DMM surgery under isoflurane anesthesia (n=6 mice/group). Knee hyperalgesia measurement was performed before injection and at 30 mins, 1 h, 2 h, and 24 h time points with a pressure application measurement device (PAM device, Ugo Basile) with the experimenter blinded to the treatment group, as previously described (PMID: 29563338). In experiment 2, a volume of 3 mL of 50 mM ND-Tam, CBD, or lidocaine formulated in 0.1% DMSO or vehicle (0.1% DMSO) was intra-articularly injected into the right knee of mice 4 weeks after DMM surgery under isoflurane anesthesia (n=5 mice/group). Knee hyperalgesia measurement was performed before injection and at 30 mins, 1 h, 2 h, 3 h, and 24 h time points with a pressure application measurement device (PAM device, Ugo Basile) with the experimenter blinded to the treatment group, as previously described (PMID: 29563338).

Immunolabeling of Neurons in Murine Knee Synovial Joints.

Naᵥ1.8-td tomato mouse knee joints were fixed in 4% paraformaldehyde, decalcified in 10% ethylenediamine tetraacetic acid (EDTA) for 2 weeks, rinsed in PBS and immersed for 72 h in 30% sucrose. The tissue was embedded in OCT compound and sections cut to 20 μm widths. For Tissue sections were rinsed in PBS and incubated for 4 hours at room temperature in primary antibody. Following PBS rinses, the slides were incubated for 2 hours at room temperature in secondary antibody. The slides were rinsed in PBS and treated with Prolong Gold anti-fade reagent. The following antibodies were used: anti-sodium channel antibody, (Sigma, S8809; anti-NeuN antibody, (Proteintech, 26975-1-AP); RFP-Booster (Chromotek, rb2AF568-50); anti-MAP2 antibody, (Proteintech, 17490-1-AP); anti-ChAT antibody (Invitrogen, PA5-29653); DAPI (Invitrogen, D1306) and cell membrane stain (Invitrogen, C₁₀₆₀₇). The images were obtained using the Nikon A1 confocal microscope.

### REFERENCES

1 Catterall, W. A. Voltage-gated sodium channels at 60: structure, function and pathophysiology. J Physiol 590, 2577-2589, doi:10.1113/jphysiol.2011.224204 (2012).
2 Goldin, A. L. Resurgence of sodium channel research. Annu Rev Physiol 63, 871-894, doi:10.1146/annurev.physiol.63.1.871 (2001).
3 Toledo-Aral, J. J. et al. Identification of PN1, a predominant voltage-dependent sodium channel expressed principally in peripheral neurons. Proc Natl Acad Sci USA 94, 1527-1532, doi:10.1073/pnas.94.4.1527 (1997).
4 Akopian, A. N., Sivilotti, L. & Wood, J. N. A tetrodotoxin-resistant voltage-gated sodium channel expressed by sensory neurons. Nature 379, 257-262, doi:10.1038/379257a0 (1996).
5 Dib-Hajj, S. D., Tyrrell, L., Black, J. A. & Waxman, S. G. NaN, a novel voltage-gated Na channel, is expressed preferentially in peripheral sensory neurons and down-regulated after axotomy. Proc Natl Acad Sci USA 95, 8963-8968, doi:10.1073/pnas.95.15.8963 (1998).
6 Ramachandra, R. & Elmslie, K. S. EXPRESS: Voltage-dependent sodium (NaV) channels in group IV sensory afferents. Mol Pain 12, doi: 10.1177/1744806916660721 (2016).
7 Cummins, T. R., Sheets, P. L. & Waxman, S. G. The roles of sodium channels in nociception: Implications for mechanisms of pain. Pain 131, 243-257, doi:10.1016/j.pain.2007.07.026 (2007).
8 Cummins, T. R., Dib-Hajj, S. D. & Waxman, S. G. Electrophysiological properties of mutant Nav1.7 sodium channels in a painful inherited neuropathy. J Neurosci 24, 8232-8236, doi:10.1523/JNEUROSCI.2695-04.2004 (2004).
9 Yang, Y. et al. Mutations in SCN9A, encoding a sodium channel alpha subunit, in patients with primary erythermalgia. J Med Genet 41, 171-174, doi:10.1136/jmg.2003.012153 (2004).
10 Bennett, D. L. & Woods, C. G. Painful and painless channelopathies. Lancet Neurol 13, 587-599, doi: 10.1016/S1474-4422(14)70024-9 (2014).
11 Chen, L. et al. Pharmacological characterization of a rat Nav1.7 loss-of-function model with insensitivity to pain. Pain 161, 1350-1360, doi: 10.1097/j.pain.0000000000001807 (2020).
12 Nahorski, M. S., Chen, Y. C. & Woods, C. G. New Mendelian Disorders of Painlessness. Trends Neurosci 38, 712-724, doi:10.1016/j.tins.2015.08.010 (2015).
13 Nahin, R. L. Estimates of pain prevalence and severity in adults: United States, 2012. J Pain 16, 769-780, doi:10.1016/j.jpain.2015.05.002 (2015).
14 Nicholson, B. Responsible prescribing of opioids for the management of chronic pain. Drugs 63, 17-32, doi:10.2165/00003495-200363010-00002 (2003).
15 Trelle, S. et al. Cardiovascular safety of non-steroidal anti-inflammatory drugs: network meta-analysis. BMJ 342, c7086, doi:10.1136/bmj.c7086 (2011).
16 Mays, T. A. Antidepressants in the management of cancer pain. Curr Pain Headache Rep 5, 227-236, doi:10.1007/s11916-001-0036-8 (2001).
17 Emery, E. C., Luiz, A. P. & Wood, J. N. Nav1.7 and other voltage-gated sodium channels as drug targets for pain relief. Expert Opin Ther Targets 20, 975-983, doi:10.1517/14728222.2016.1162295 (2016).
18 Shen, H., Liu, D., Wu, K., Lei, J. & Yan, N. Structures of human Naᵥ1.7 channel in complex with auxiliary subunits and animal toxins. Science 363, 1303-1308, doi:10.1126/science.aaw2493 (2019).
19 Cao, E., Liao, M., Cheng, Y. & Julius, D. TRPV1 structures in distinct conformations reveal activation mechanisms. Nature 504, 113-118, doi: 10.1038/nature12823 (2013).
20 Jiang, D. et al. Structural basis for voltage-sensor trapping of the cardiac sodium channel by a deathstalker scorpion toxin. Nat Commun 12, 128, doi:10.1038/s41467-020-20078-3 (2021).
21 Wisedchaisri, G. et al. Structural Basis for High-Affinity Trapping of the NaV1.7 Channel in Its Resting State by Tarantula Toxin. Mol Cell 81, 38-48 e34, doi: 10.1016/j.molcel.2020.10.039 (2021).
22 Yan, Z. et al. Structure of the Nav1.4-beta1 Complex from Electric Eel. Cell 170, 470-482 e411, doi:10.1016/j.cell.2017.06.039 (2017).
23 Bagneris, C. et al. Prokaryotic NavMs channel as a structural and functional model for eukaryotic sodium channel antagonism. Proc Natl Acad Sci USA 111, 8428-8433, doi:10.1073/pnas.1406855111 (2014).
24 Zanatta, G. et al. Valproic acid interactions with the NavMs voltage-gated sodium channel. Proc Natl Acad Sci USA, doi:10.1073/pnas.1909696116 (2019).
25 Sait, L. G. et al. Cannabidiol interactions with voltage-gated sodium channels. Elife 9, doi:10.7554/eLife.58593 (2020).
26 Xu, H. et al. Structural Basis of Nav1.7 Inhibition by a Gating-Modifier Spider Toxin. Cell 176, 702-715 e714, doi:10.1016/j.cell.2018.12.018 (2019).
27 Sula, A. et al. A tamoxifen receptor within a voltage-gated sodium channel. Mol Cell, doi:10.1016/j.molcel.2020.12.048 (2021).
28 Payandeh, J., Scheuer, T., Zheng, N. & Catterall, W. A. The crystal structure of a voltage-gated sodium channel. Nature 475, 353-358, doi: 10.1038/nature10238 (2011).
29 Ren, D. et al. A prokaryotic voltage-gated sodium channel. Science 294, 2372-2375, doi:10.1126/science.1065635 (2001).
30 Vien, T. N. & DeCaen, P. G. Biophysical Adaptations of Prokaryotic Voltage-Gated Sodium Channels. Curr Top Membr 78, 39-64, doi: 10.1016/bs.ctm.2015.12.003 (2016).
31 Jordan, V. C. Fourteenth Gaddum Memorial Lecture. A current view of tamoxifen for the treatment and prevention of breast cancer. Br J Pharmacol 110, 507-517, doi:10.1111/j.1476-5381.1993.tb13840.x (1993).
32 Radin, D. P. & Patel, P. Delineating the molecular mechanisms of tamoxifen's oncolytic actions in estrogen receptor-negative cancers. Eur J Pharmacol 781, 173-180, doi:10.1016/j.ejphar.2016.04.017 (2016).
33 Crewe, H. K., Notley, L. M., Wunsch, R. M., Lennard, M. S. & Gillam, E. M. Metabolism of tamoxifen by recombinant human cytochrome P450 enzymes: formation of the 4-hydroxy, 4′-hydroxy and N-desmethyl metabolites and isomerization of trans-4-hydroxytamoxifen. Drug Metab Dispos 30, 869-874, doi:10.1124/dmd.30.8.869 (2002).
34 Jordan, V. C. New insights into the metabolism of tamoxifen and its role in the treatment and prevention of breast cancer. Steroids 72, 829-842, doi:10.1016/j.steroids.2007.07.009 (2007).
35 Katzenellenbogen, B. S., Norman, M. J., Eckert, R. L., Peltz, S. W. & Mangel, W. F. Bioactivities, estrogen receptor interactions, and plasminogen activator-inducing activities of tamoxifen and hydroxy-tamoxifen isomers in MCF-7 human breast cancer cells. Cancer Res 44, 112-119 (1984).
36 Sula, A. et al. A tamoxifen receptor within a voltage-gated sodium channel. Mol Cell 81, 1160-1169 e1165, doi:10.1016/j.molcel.2020.12.048 (2021).
37 Lin, Z., Santos, S., Padilla, K., Printzenhoff, D. & Castle, N. A. Biophysical and Pharmacological Characterization of Nav1.9 Voltage Dependent Sodium Channels Stably Expressed in HEK-293 Cells. PLoS One 11, e0161450, doi:10.1371/journal.pone.0161450 (2016).
38 Yang, Y. C. & Kuo, C. C. An inactivation stabilizer of the Na+ channel acts as an opportunistic pore blocker modulated by external Na+. J Gen Physiol 125, 465-481, doi: 10.1085/jgp.200409156 (2005).
39 Castaneda-Castellanos, D. R., Nikonorov, I., Kallen, R. G. & Recio-Pinto, E. Lidocaine stabilizes the open state of CNS voltage-dependent sodium channels. Brain Res Mol Brain Res 99, 102-113, doi:10.1016/s0169-328x(01)00340-0 (2002).
40 Ghovanloo, M. R. et al. Inhibitory effects of cannabidiol on voltage-dependent sodium currents. J Biol Chem 293, 16546-16558, doi:10.1074/jbc.RA118.004929 (2018).
41 Willow, M., Gonoi, T. & Catterall, W. A. Voltage clamp analysis of the inhibitory actions of diphenylhydantoin and carbamazepine on voltage-sensitive sodium channels in neuroblastoma cells. Mol Pharmacol 27, 549-558 (1985).
42 Pammolli, F., Magazzini, L. & Riccaboni, M. The productivity crisis in pharmaceutical R&D. Nat Rev Drug Discov 10, 428-438, doi:10.1038/nrd3405 (2011).
43 Chaplan, S. R., Eckert, I. W. & Carruthers, N. I. in Translational Pain Research: From Mouse to Man Frontiers in Neuroscience (eds L. Kruger & A. R. Light) (2010).
44 Waring, M. J. et al. An analysis of the attrition of drug candidates from four major pharmaceutical companies. Nat Rev Drug Discov 14, 475-486, doi: 10.1038/nrd4609 (2015).
45 Woolf, C. J. Capturing Novel Non-opioid Pain Targets. Biol Psychiatry 87, 74-81, doi:10.1016/j.biopsych.2019.06.017 (2020).
46 Palermo, A. F. et al. Incorporation of histone deacetylase inhibitory activity into the core of tamoxifen—A new hybrid design paradigm. Bioorganic & Medicinal Chemistry 26, 4428-4440, doi:https://doi.org/10.1016/j.bmc.2018.07.026 (2018).
47 Fauq, A. H., Maharvi, G. M. & Sinha, D. A convenient synthesis of (Z)-4-hydroxy-N-desmethyltamoxifen (endoxifen). Bioorganic & Medicinal Chemistry Letters 20, 3036-3038, doi:https://doi.org/10.1016/j.bmcl.2010.03.117 (2010).

1. A method for treating pain in a subject in need thereof, the method comprising administering to the subject an effective amount of a compound having a formula as follows or a suitable pharmaceutical salt thereof: 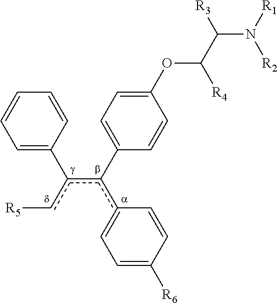
wherein:
R¹ is selected from hydrogen, alkyl, alkoxy, hydroxy, and halogen;
R² is selected from hydrogen, alkyl, alkoxy, hydroxy, and halogen;
R³ is selected from hydrogen, alkyl, alkoxy, hydroxy, halogen, and amino;
R⁴ is selected from hydrogen, alkyl, alkoxy, hydroxy, halogen, and amino;
R⁵ is selected from hydrogen, alkyl, alkoxy, hydroxy, halogen, and amino; and
R⁶ is selected from hydrogen and hydroxy when there are single bonds between C^{α}-C^{β} and C^{γ}-C^{δ} and a double bond between C^{β}-C^{γ} or R⁶ is oxo when there are double bonds between C^{α}-C^{β} and C^{γ}-C^{δ} and a single bond between C^{β}-C^{γ}.

2. The method of claim 1, wherein the compound has a formula as follows or a suitable pharmaceutical salt thereof.

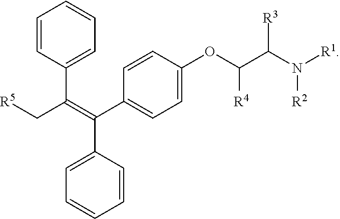

3. The method of claim 1, wherein the subject has nociceptive pain.

4. The method of claim 1, wherein the subject has inflammatory pain.

5. The method of claim 1, wherein the subject has neuropathic pain.

6. The method of claim 1, wherein the subject has chronic pain.

7. The method of claim 1, wherein the subject has pain associated with osteoarthritis.

8. The method of claim 1, wherein the subject has pain associated with an activated voltage-gated sodium channel (Naᵥs).

9. The method of claim 1, wherein the compound binds to a voltage-gated sodium channel (Naᵥ) and has a dissociation constant (K_{d}) less than 50 nM.

10. The method of claim 1, wherein the compound inhibits the activity of a voltage-gated sodium channel (Naᵥ) and has a half-maximal inhibitory concentration (IC₅₀) of less than about 200 nM.

11. The method of claim 10, wherein the Naᵥ is selected from Na 1.1, Nav1.2, Naᵥ1.3, Naᵥ1.4, Naᵥ1.5, Naᵥ1.6, Naᵥ1.7, Naᵥ1.8, and Naᵥ1.9.

12. The method of claim 1, wherein the compound is administered orally, topically, or intravenously.

13. The method of claim 1, wherein the subject is administered a dose of the compound that is effective for achieving a concentration of the compound at the site of action of at least 1 nM.

14. The method of claim 1, wherein the subject is administered a daily oral dose of the compound of less than 300 mg.

15. The method of claim 1, wherein the subject is administered a daily dose of less than 10 mg/kg.

16. The method of claim 1, wherein the compound does not bind to the estrogen receptor or the compound binds to the estrogen receptor and has a dissociation constant (K_{d}) for the estrogen receptor which greater than 100 nM.

17. The method of claim 1, wherein R¹ and R² are independently selected from hydrogen and methyl, R³ is hydrogen, and R⁴ is hydrogen.

18. The method of claim 1, wherein the compound is tamoxifen, N-desmethyltamoxifen, 4-hydroxytamoxifen, N-desmethyl-4-hydroxytamoxifen, or 4-hydroxytamoxifen quinone methide.

19. The method of claim 1, wherein the compound is N-desmethyltamoxifen.

20. The method of claim 1, wherein the compound is not tamoxifen, N-desmethyltamoxifen, 4-hydroxytamoxifen, tamoxifen, N-desmethyl-4-hydroxytamoxifen, or 4-hydroxytamoxifen quinone methide.

21. A compound having a formula as follows or a suitable pharmaceutical salt thereof: 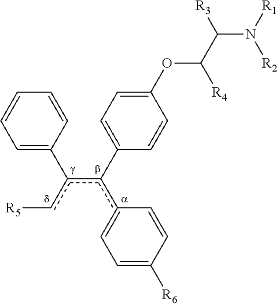
wherein:
R¹ is selected from hydrogen, alkyl, alkoxy, hydroxy, and halogen;
R² is selected from hydrogen, alkyl, alkoxy, hydroxy, and halogen;
R³ is selected from hydrogen, alkyl, alkoxy, hydroxy, halogen, and amino;
R⁴ is selected from hydrogen, alkyl, alkoxy, hydroxy, halogen, and amino; and
R⁵ is selected from hydrogen, alkyl, alkoxy, hydroxy, halogen, and amino; and
R⁶ is selected from hydrogen and hydroxy when there are single bonds between C^{α}-C^{β} and C^{γ}-C^{δ} and a double bond between C^{β}-C^{γ} or R⁶ is oxo when there are double bonds between C^{α}-C^{β} and C^{γ}-C^{δ} and a single bond between C^{β}-C^{γ},
with the proviso that the compound is not any of tamoxifen, N-desmethyltamoxifen, 4-hydroxytamoxifen, N-desmethyl-4-hydroxytamoxifen, and 4-hydroxytamoxifen quinone methide.

22. A pharmaceutical composition comprising an effective amount of the compound according to claim 1 to treat pain in a subject in need thereof and a suitable pharmaceutical carrier, excipient, or solvent.
